(19) United States
(12) Patent Application Publication
(10) Pub. No.: US 2023/0279006 A1
(43) Pub. Date: Sep. 7, 2023

(54) NOVEL COMPOUNDS AND METHODS FOR INCREASING KLOTHO GENE EXPRESSION

(71) Applicant: KLOTHO THERAPEUTICS, INC., Las Vegas, NV (US)

(72) Inventors: James Plante, Las Vegas, NV (US); William Ramage, Del Mar, CA (US); Christopher Smith, San Diego, CA (US); MICHAEL BISHOP, SAN DIEGO, CA (US)

(21) Appl. No.: 18/015,040

(22) PCT Filed: Jul. 8, 2021
(86) PCT No.: PCT/US2021/040937

§ 371 Date: Jan. 6, 2023

Related U.S. Application Data
(60) Provisional application No. 63/049,614, filed on Jul. 8, 2020.

(51) Int. Cl. C07D 473/34; C07D 239/48; C07D 239/94

(52) U.S. Cl. CPC C07D 473/34; C07D 239/48; C07D 239/94

(57) ABSTRACT

Novel compounds and compositions including the same and methods of manufacturing and using the same, particularly for increasing klotho gene expression, and more particularly for increasing circulating or soluble Klotho protein levels through increasing klotho gene expression.

### BACKGROUND

### 1. Technical Field

The present disclosure relates to novel compounds and compositions comprising the same and to methods of manufacturing and using the same, particularly for increasing Klotho gene expression.

### 2. Related Technology

Klotho (or alpha-Klotho, α-Klotho, etc.) is a recently characterized protein encoded by the KL (or klotho) gene, located on human chromosome 13. Two transcripts that arise from a single klotho gene through alternative RNA splicing have been identified. See FIGS. 1 and 2. The first transcript is predicted to encode Klotho isoform 1 - a full-length, 1,012 amino acid, single-pass transmembrane-membrane protein, with a short cytoplasmic tail (human residues 1003-1012), a transmembrane (TM) domain (human residues 982-1002), and extracellular region or domain (human residues 1-981) comprising two largely homologous (internal repeat) domains (termed KL1 (human residues 56-506, which is 450 residues long) and KL2 (human residues 515-953, which is 438 residues long), which each share 20%-40% amino acid sequence homology to β-glucosidases, but may lack similar levels of glucosidase catalytic activity), and a signal sequence (SS) domain (human residues 1-33). The SS, KL1, and KL2 domain-containing extracellular region (human residues 1-981) may be enzymatically cleaved by α/β-secretases, and released into the circulatory stream as a 130 kDa circulating protein, termed soluble klotho (or sKlotho, s-Klotho, alpha soluble-Klotho, etc.). The extracellular region can also be cleaved into separate 68 kDa protein (KL1 + SS) and 64 kDa protein (KL2).

The second transcript, a splicing variant of alpha-klotho mRNA, encodes a second isoform of Klotho protein corresponding mainly to the KL1 domain. The internal splice donor site is thought to be located in exon 3 of the klotho gene. The resultant alternatively spliced transcript contains a 50 bp insertion after exon 3, with an in-frame translation stop codon at the end thereof. The expressed protein product is secreted into the circulation and is termed secreted Klotho (or Klotho isoform 2), which differs from the canonical sequence of isoform 1 at amino acid residues 535-549: DTTLSQFTDLNVYLW → SQLTKPISSLTKPYH, and with amino acid residues 550-1012 missing.

Accordingly, there may be a number of different Klotho proteins in the circulation at any given time, depending on gene expression, RNA splicing, and enzymatic cleavage. Despite the existence of various forms of alpha-Klotho protein, only the full length, membrane-bound, isoform 1 is known to form a complex with fibroblast growth factor (FGF) receptors and functions as an obligatory co-receptor for FGF23 - a bone-derived hormone that induces phosphate excretion into urine and which has a regulatory role on Pᵢ and vitamin D metabolism.

Klotho is highly expressed in the kidney, brain, and to a lesser extent in other organs, and may also be found in the cerebrospinal fluid and urine of mammals. Circulating levels of soluble Klotho proteins in mammals are thought to decrease with age. In addition, Klotho-deficient mice exhibit accelerated aging phenotypes, whereas over-expression of klotho in mice has been shown to extend lifespan. In addition, Klotho has been implicated in a number of cellular processes related to aging. In light of the foregoing, a developing hypothesis states that soluble Klotho may function as an anti-aging compound in the human body.

The Klotho gene was originally identified as a putative aging-suppressor gene in mice that extended life span when overexpressed and induced a premature aging syndrome when disrupted. Subsequently, the Klotho gene was found to be involved in numerous aging-associated pathologies, including chronic kidney disease, diabetes, cancers, cognitive decline, sarcopenia and cardiovascular diseases. For example, a deficiency of the Klotho gene can cause arterial stiffness. Further, Klotho protein levels decrease with age while the prevalence of arterial stiffness and hypertension increase with age. For example, at age 70 years, the serum level of Klotho protein in a human is only about one half of what it was at age 40 years. Moreover, the serum Klotho protein level is significantly decreased in humans with arterial stiffness and chronic kidney diseases.

Aging is an inevitable and progressive biological process resulting in dysfunction and destruction of almost all tissues and organs, ultimately resulting in death. The aging of the human body, for instance, is associated with the decline of cellular function, which can lead to the development of a variety of diseases. Aging is thought to be driven by a tightly regulated and complex interplay between genetic, epigenetic, and acquired factors and is typically characterized by an increase in senescence, a quantitative and qualitative decrease in stem cells, and abnormal structure at tissue levels. For instance, an increase in DNA methylation in the promoter region of a gene diminishes the promoter activity and gene transcription. DNA demethylation is a physiological process that maintains transcriptional activity of genes, while DNA methylation is increased with age and the prevalence of arterial stiffness and hypertension are also increased with age. Physiologically, an appropriate methylation level is maintained by the balanced methylase and demethylase activity.

As the so-called “baby boomers” generation continues to advance in age, the population of aging individuals (e.g., age 60-65) is rapidly increasing globally. The increased demand for health care for this aging population places significant financial burden on any healthcare system. Molecular compounds (or so-called “small molecules”) provide promising therapeutic agents to counter age-related health conditions. Developing strategies and health intervention methods based on the production and purification of compounds that increase klotho gene expression, and the administration of such compounds to subjects may help to ameliorate this situation and the problems associated therewith. Developing strategies and health intervention methods based on the administration of small molecules that increase Klotho protein levels through increasing klotho gene expression to subjects, especially humans and/or within an increasing aging population, may help to ameliorate this situation.

Currently, there is not a commercial small molecule product or treatment method for increasing Klotho protein levels through increasing klotho gene expression, especially products and methods approved by the U.S. Food and Drug Administration (FDA). To date, all relevant data is connected to pre-clinical research studies in animal models. For example, King et al. published the Identification of novel small molecules that elevate Klotho expression (Biochem J. 2012 January 1; 441(1): 453-461), Jung et al. published the Induction of anti-aging gene klotho with a small chemical compound that demethylates CpG islands (Oncotarget, 2017, Vol. 8, (No. 29), pp: 46745-46755), Chen and Sun published the Activation of DNA demethylases attenuates aging-associated arterial stiffening and hypertension (Aging Cell. 2018; e12762), and US 2018/0338951 discloses Treatments for arterial stiffening, hypertension and anti-aging. However, each of the foregoing references, the entirety of each of which is incorporated by reference herein, falls short of providing a compound for FDA approved administration. For example, King et al. teaches a Compound H, which is deficient in therapeutic efficacy.

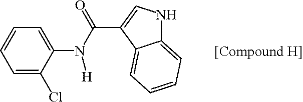 [SMILES: O=C(Nc1ccccc1Cl)c1c[nH]c2ccccc12]

Accordingly, there are a number of short-comings in the art that can be addressed by the development, production, manufacture, and administration of small molecules or compounds for increasing Klotho protein levels through increasing klotho gene expression.

### BRIEF SUMMARY

Embodiments of the present disclosure solve one or more of the foregoing or other problems in the art with compounds (e.g., small molecules) and compositions including the same for increasing klotho gene expression, particularly for increasing circulating and/or soluble Klotho protein levels through increasing klotho gene expression, and methods of manufacturing and using the same.

Illustratively, the novel compounds are, or function as, therapeutic agents that activate, enhance, or increase klotho gene expression, thereby increasing circulating and/or soluble Klotho protein levels in mammals. Accordingly, some embodiments include a method of increasing klotho gene expression, or a method for increasing circulating and/or soluble Klotho protein levels through increasing klotho gene expression, the methods comprising administering a composition of the present disclosure to a mammalian subject.

Illustrative embodiments of the present disclosure include a compound according to Formula I:

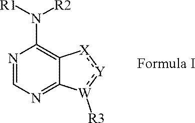

or a stereoisomer, tautomer or pharmaceutically acceptable salt thereof.

In various embodiments, X is (selected from (the group consisting of)) N, S, or C—R₄, wherein R₄ is (selected from (the group consisting of)) H or C1-C5 alkyl. In some embodiments, X is N. In some embodiments, X is S. In some embodiments, X is CH. In some embodiments, R₄ is H. In some embodiments, R₄ is C1-C5 alkyl.

In various embodiments, Y is (selected from (the group consisting of)) N or C—R₄, wherein R₄ is (selected from (the group consisting of)) H or C1-C5 alkyl. In some embodiments, Y is N. In some embodiments, Y is CH. In some embodiments, R₄ is H. In some embodiments, R₄ is C1-C5 alkyl.

In various embodiments, the bond between X-Y is a single bond or a double bond. In some embodiments, the bond between X-Y is a single bond. In some embodiments, the bond between X-Y is a double bond.

In various embodiments, W is (selected from (the group consisting of)) N, S, or C. In some embodiments, W is N. In some embodiments, W is S. In some embodiments, W is C.

In various embodiments, the bond between Y-W is a single bond or a double bond. In some embodiments, the bond between Y-W is a single bond. In some embodiments, the bond between Y-W is a double bond.

In various embodiments, R₁ is (selected from (the group consisting of)) H, CH₃, or, together with R₂, forms substituted or unsubstituted heterocyclic amine. In some embodiments, R₁ is H. In some embodiments, R₁ is CH₃. In various embodiments, R₁, together with R₂, forms substituted or unsubstituted heterocyclic amine.

In various embodiments, R₂ is (selected from (the group consisting of)):

(CH₂)_{Z}—(CR₅R₆)_{V}—R₇, wherein Z is an integer from 0-2, V is an integer from 0-2, R₅ is (selected from (the group consisting of)) H or CH₃, R₆ is (selected from (the group consisting of)) H or CH₃; and R₇ is (selected from (the group consisting of)):
(i) saturated or unsaturated C3-C8 substituted or unsubstituted cycloalkyl or bicycloalkyl (e.g., bicyclo octane, preferably, bicyclo(2.2.2)octane), optionally substituted at one or more (ring) positions and each (ring) substituent is (selected from (the group consisting of)) halo, aryl (phenyl or benzyl), or branched or unbranched C1-C3 alkyl;
(ii) substituted or unsubstituted aryl (phenyl or benzyl), optionally substituted at one or more (1 or 2) (ring) positions and each (ring) substituent is (selected from (the group consisting of))halo or branched or unbranched C1-C3 alkyl; and/or
(iii) branched or unbranched C1-C3 alkyl (e.g., isopropyl);

C3-C7 substituted or unsubstituted cycloalkyl (e.g., 2-phenylcyclopropyl), optionally substituted at one or more (ring) positions and each (ring) substituent is (selected from (the group consisting of)) substituted or unsubstituted aryl (phenyl or benzyl), branched or unbranched C1-C3 substituted or unsubstituted alkyl; and/or

together with R₁, forms substituted or unsubstituted heterocyclic amine.

In some embodiments, Y and R₃ do not form heterocyclic amine. In some embodiments, R₂ is not C3 cycloalkyl, preferably substituted with aryl (or phenyl or benzyl), preferably 2-phenylcyclopropyl. In some embodiments, R₂ is not aryl (or phenyl or benzyl), preferably substituted at one or more (ring) positions with halo, preferably chloro, more preferably 3-chlorobenzyl or 2,3-dichlorobenzyl. In some embodiments, R₂ is not:

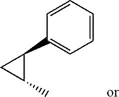

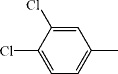

In various embodiments, R₃ is (selected from (the group consisting of)): nothing, H, alkyl, cycloalkyl, aryl (phenyl or benzyl), nitrile, (CH₂)_{Z}CN, wherein Z is an integer from 1-3, branched or unbranched C1-C3 substituted or unsubstituted alkyl, C3-C7 substituted or unsubstituted cycloalkyl, aryl (phenyl or benzyl) optionally substituted at one or more (ring) positions and each (ring) substituent is (selected from (the group consisting of)) branched or unbranched C1-C3 substituted or unsubstituted alkyl, halo, or nitrile. In some embodiments, R₃ is nothing. In some embodiments, R₃ is H. In some embodiments, R₃ is alkyl. In some embodiments, R₃ is cycloalkyl. In some embodiments, R₃ is aryl (phenyl or benzyl). In some embodiments, R₃ is nitrile. In some embodiments, R₃ is (CH₂)_{Z}CN, wherein Z is an integer from 1-3. In some embodiments, R₃ is branched or unbranched C1-C3 substituted or unsubstituted alkyl. In some embodiments, R₃ is C3-C7 substituted or unsubstituted cycloalkyl. In some embodiments, R₃ is aryl (phenyl or benzyl) substituted at one or more (ring) positions and each (ring) substituent is (selected from (the group consisting of)) branched or unbranched C1-C3 substituted or unsubstituted alkyl, halo, or nitrile. In some embodiments, R₃ is aryl (phenyl or benzyl) substituted at one or more (ring) positions with branched or unbranched C1-C3 substituted or unsubstituted alkyl, halo, or nitrile.

In some embodiments, R₁ is H and R₂ is (CH₂)_{Z}—(CR₅R₆)_{V}—R₇, wherein Z is an integer from 1-2, V is 0, and R₇ is (selected from (the group consisting of):
saturated or unsaturated C4-C7 cycloalkyl, optionally substitute at one or more (ring) positions with one or more methyl, preferably saturated C4-C6 cycloalkyl, optionally substitute at one or more (ring) positions with one or more methyl, more preferably unsubstituted saturated C4-C6 cycloalkyl or saturated C6 cycloalkyl optionally substituted at one or more (ring) position with one or more methyl or fluoro, still more preferably monounsaturated C5-C7 cycloalkyl, preferably 2-(1-cycloalkenyl or 4-(1-cycloalkenyl, more preferably 2-(1-cyclopentenyl), 2-(1-cyclohexenyl), 2-(1-cycloheptenyl), 4-(1-cyclopentenyl), 4-(1-cyclohexenyl), or 4-(1-cycloheptenyl);
bicycloalkyl, preferably bicyclo octane, more preferably, bicyclo(2.2.2)octane;
aryl (or phenyl or benzyl), preferably substituted at one or more (ring) positions with halo, preferably chloro, more preferably 3-chlorobenzyl or 2,3-dichlorobenzyl;
isopropyl; and/or
C3 cycloalkyl, preferably substituted with aryl (or phenyl or benzyl), preferably 2-phenylcyclopropyl.

In some embodiments, R1 is H and R2 is (CH₂)_{z}—(CR₅R₆)ᵥ—R₇, wherein Z is 1, V is 1, R₅ is CH₃, R₆ is CH₃, and R₇ is monounsaturated cyclohexyl, preferably 2-(1-cyclohexenyl).

In some embodiments, R₁ is CH₃ and R₂ is (CH₂)_{z}—(CR₅R₆)ᵥ—R₇, Z is 2, V is 0, and R₇ is cyclohexyl.

In some embodiments, R₁ together with R₂, forms substituted or unsubstituted heterocyclic amine, preferably, substituted azepane, more preferably 4,4-diethylazepane. In some embodiments, R₁ and R₂ form a substituted or unsubstituted, saturated or unsaturated heterocyclic amine, preferably a substituted azepane, aziridine, azetidine, pyrrolidine, piperidine, or azocane, more preferably azepane, still more preferably 4,4-diethyl azepane.

In some embodiments:
X is N, Y is C—R₄, R₄ is H, W is N, the bond between X-Y is a double bond, and the bond between Y-W is a single bond, and wherein R₃ is preferably alkyl or cycloalkyl, more preferably ethyl or cyclopropyl;
X is C—R₄, Y is C—R₄, R₄ is H, W is N, the bond between X-Y is a double bond, and the bond between Y-W is a single bond, and preferably, wherein R₃ is H;
X is C—R₄, R₄ is H, Y is N, W is N, the bond between X-Y is a double bond, and the bond between Y-W is a single bond, and preferably, wherein R₃ is preferably (i) alkyl, more preferably methyl, (ii) substituted or unsubstituted aryl, phenyl or benzyl, still more preferably fluorophenyl or fluorobenzyl, or (iii) nitrile, preferably ethanenitrile or ethyl cyanide;
X is N or C—R₄, Y is C—R₄, each R₄ is, independently, H or CH₃, W is S, the bond between X-Y is a double bond, and the bond between Y-W is a single bond, and wherein R₃ is nothing;
X is C—R₄, Y is C—R₄, each R₄ is, independently, H or CH₃, W is S, the bond between X-Y is a double bond, and the bond between Y-W is a single bond, and wherein R₃ is nothing;
X is N, Y is C—R₄, R₄ is H, W is S, the bond between X-Y is a doble bond, and the bond between Y-W is a single bond, and wherein R₃ is nothing;
X is N, Y is C—R₄, R₄ is H, W is N, the bond between X-Y is a double bond, and the bond between Y-W is a single bond, and wherein R₃ is preferably C2-C3 alkyl or cycloalkyl, preferably, ethyl or cyclopropyl; or
X is S, Y is C—R₄, R₄ is H, W is C, the bond between X-Y is a single bond, and the bond between Y-W is a double bond, and wherein R₃ is preferably H.

In some embodiments, X is N, Y is C—R₄, R₄ is H, W is N, the bond between X-Y is a double bond, and the bond between Y-W is a single bond, and wherein R₃ is preferably alkyl or cycloalkyl, more preferably ethyl or cyclopropyl.

In some embodiments, X is C—R₄, Y is C—R₄, R₄ is H, W is N, the bond between X-Y is a double bond, and the bond between Y-W is a single bond, and preferably, wherein R₃ is H.

In some embodiments, X is C—R₄, R₄ is H, Y is N, W is N, the bond between X-Y is a double bond, and the bond between Y-W is a single bond, and preferably, wherein R₃ is preferably (selected from (the group consisting of)) (i) alkyl, more preferably methyl, (ii) substituted or unsubstituted aryl, phenyl or benzyl, still more preferably fluorophenyl or fluorobenzyl, or (iii) nitrile, preferably ethanenitrile or ethyl cyanide.

In some embodiments, X is N or C—R₄, Y is C—R₄, each R₄ is, independently, H or CH₃, W is S, the bond between X-Y is a double bond, and the bond between Y-W is a single bond, and wherein R₃ is nothing.

In some embodiments, X is C—R₄, Y is C—R₄, each R₄ is, independently, H or CH₃, W is S, the bond between X-Y is a double bond, and the bond between Y-W is a single bond, and wherein R₃ is nothing.

In some embodiments, X is N, Y is C—R₄, R₄ is H, W is S, the bond between X-Y is a doble bond, and the bond between Y-W is a single bond, and wherein R₃ is nothing.

In some embodiments, X is N, Y is C—R₄, R₄ is H, W is N, the bond between X-Y is a double bond, and the bond between Y-W is a single bond, and wherein R₃ is preferably (selected from (the group consisting of)) C2-C3 alkyl or cycloalkyl, preferably, ethyl or cyclopropyl.

In some embodiments, X is S, Y is C—R₄, R₄ is H, W is C, the bond between X-Y is a single bond, and the bond between Y-W is a double bond, and wherein R₃ is preferably H.

In some embodiments, X is N, Y is C—R₄, R₄ is H, W is N, the bond between X-Y is a double bond, the bond between Y-W is a single bond, R₁ is H, R₂ is (CH₂)_{Z}—(CR₅R₆)_{V}—R₇, wherein Z is 1, V is 0, and R₇ is (selected from (the group consisting of)) unsubstituted monounsaturated C6 cycloalkyl or unsubstituted cyclohexenyl, preferably 4-(1-cyclohexenyl), more preferably R-4-(1-cyclohexenyl) or S-4-(1-cyclohexenyl), and R₃ is cyclopropyl.

In some embodiments, X is N, Y is C—R₄, R₄ is H, W is N, the bond between X-Y is a double bond, and the bond between Y-W is a single bond, R₃ is (selected from (the group consisting of)) alkyl or cycloalkyl, preferably ethyl or cyclopropyl, R₁ is H and R₂ is (CH₂)_{Z}—(CR₅R₆)_{V}—R₇, wherein Z is an integer from 1-2, V is 0, and R₇ is (selected from (the group consisting of)):
unsubstituted saturated C4-C6 cycloalkyl;
saturated C6 cycloalkyl substituted at one (ring) positions with fluoro, methyl, or dimethyl, preferably 1-fluorocyclohexyl, 1-methylcyclohexyl, or 4,4-dimethylcyclohexyl;
unsubstituted monounsaturated C5-C7 cycloalkyl, preferably 2-(1-cycloalkenyl or 4-(1-cycloalkenyl, more preferably 2-(1-cyclopentenyl), 2-(1-cyclohexenyl), 2-(1-cycloheptenyl), 4-(1-cyclopentenyl), 4-(1-cyclohexenyl), or 4-(1-cycloheptenyl);
bicyclo octane, more preferably, bicyclo(2.2.2)octane;
aryl (or phenyl or benzyl) substituted at one or two (ring) positions with halo, preferably chloro, more preferably 3-chlorobenzyl or 2,3-dichlorobenzyl;
isopropyl; and/or
C3 cycloalkyl substituted with aryl (or phenyl or benzyl), preferably 2-phenylcyclopropyl.

Illustrative embodiments of the present disclosure include a compound according to Formula Ia:

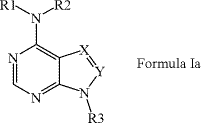

or a stereoisomer, tautomer or pharmaceutically acceptable salt thereof, wherein X, Y, R₁, R₂, and R₃ are as described for Formula I.

Illustrative embodiments of the present disclosure include a compound according to Formula II:

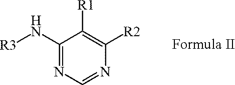

or a stereoisomer, tautomer or pharmaceutically acceptable salt thereof.

In various embodiments, R₁ is (selected from (the group consisting of) H or NH₂. In some embodiments, R₁ is H. In some embodiments, R₁ is NH₂.

In various embodiments, R₂ is (selected from (the group consisting of) saturated or unsaturated, heterocyclic amine or heterocyclic diamine. In some embodiments, R₂ is saturated or unsaturated heterocyclic amine. In some embodiments, R₂ is saturated or unsaturated heterocyclic diamine.

In some embodiments, R₂ is saturated heterocyclic amine, preferably N-pyrrolidine or N-piperidine, or unsaturated heterocyclic diamine, preferably N-pyrazole.

In some embodiments, when R₁ is H, then R₂ is N-pyrrolidine or N-piperidine.

In some embodiments, when R₁ is NH₂, then R₂ is N-pyrazole.

In various embodiments, R₃ is (CH₂)₂—R₇, wherein R₇ is unsaturated cycloalkyl.

In some embodiments, R₇ is monounsaturated C6-C7 cycloalkyl, preferably cyclohexenyl, more preferably 2-(1-cyclohexenyl), or cycloheptenyl, more preferably 2-(1-cycloheptenyl).

In some embodiments, the compound of Formula II is selected from the group consisting of Formulas IIa-IId:

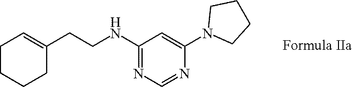 [SMILES: C1=C(CCNc2cc(N3CCCC3)ncn2)CCCC1]

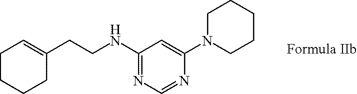 [SMILES: C1=C(CCNc2cc(N3CCCCC3)ncn2)CCCC1]

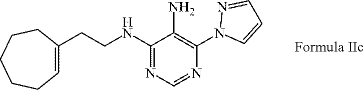 [SMILES: Nc1c(NCCC2=CCCCCC2)ncnc1-n1cccn1]

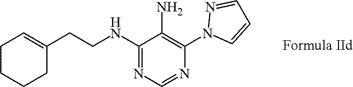 [SMILES: Nc1c(NCCC2=CCCCC2)ncnc1-n1cccn1]

or a stereoisomer, tautomer or pharmaceutically acceptable salt thereof.

Illustrative embodiments of the present disclosure include a compound according to Formula III:

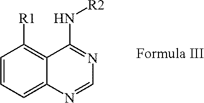

or a stereoisomer, tautomer or pharmaceutically acceptable salt thereof, wherein R₁ is halo; and R₂ is (CH₂)₂—R₇, wherein R₇ is unsaturated cycloalkyl. In some embodiments, R₁ is fluoro and R₇ is unsaturated cycloalkyl, preferably monounsaturated cycloalkyl, more preferably cyclohexenyl, more preferably 2-(1-cyclohexenyl), still most preferably, wherein the compound is according to Formula IIIa:

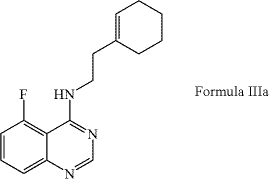 [SMILES: Fc1cccc2ncnc(NCCC3=CCCCC3)c12]

In some embodiments, the compound according to Formula I can be one of Compounds 1-49 of Table 1.

TABLE 1

Nonlimiting examples of illustrative compounds

| Compound Number | Structure | Mol. Wt. | Molecular Formula |
|---|---|---|---|
| 1 | 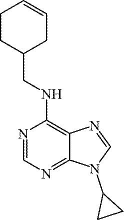 [SMILES: C1=CCC(CNc2ncnc3c2ncn3C2CC2)CC1] | 269.4 | C15H19N5 |
| 2 | 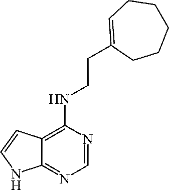 [SMILES: C1=C(CCNc2ncnc3[nH]ccc23)CCCCC1] | 256.4 | C15H20N4 |
| 3 | 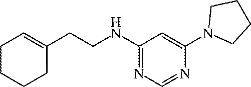 [SMILES: C1=C(CCNc2cc(N3CCCC3)ncn2)CCCC1] | 272.4 | C16H24N4 |
| 4 | 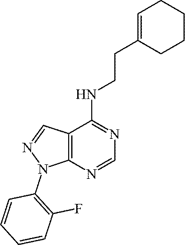 [SMILES: Fc1ccccc1-n1ncc2c(NCCC3=CCCCC3)ncnc21] | 337.4 | C19H20N5F |
| 5 | 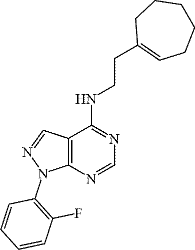 [SMILES: Fc1ccccc1-n1ncc2c(NCCC3=CCCCCC3)ncnc21] | 351.4 | C20H22N5F |
| 6 | 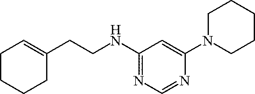 [SMILES: C1=C(CCNc2cc(N3CCCCC3)ncn2)CCCC1] | 286.4 | C17H26N4 |
| 7 | 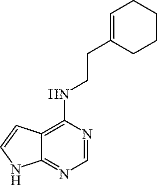 [SMILES: C1=C(CCNc2ncnc3[nH]ccc23)CCCC1] | 242.3 | C14H18N4 |
| 8 | 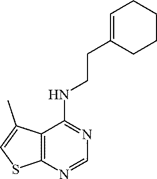 [SMILES: Cc1csc2ncnc(NCCC3=CCCCC3)c12] | 273.4 | C15H19N3S |
| 9 | 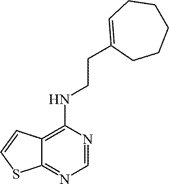 [SMILES: C1=C(CCNc2ncnc3sccc23)CCCCC1] | 273.4 | C15H19N3S |
| 10 | 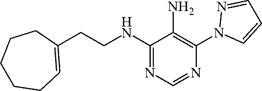 [SMILES: Nc1c(NCCC2=CCCCCC2)ncnc1-n1cccn1] | 298.4 | C16H22N6 |
| 11 | 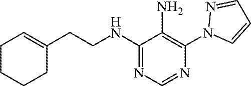 [SMILES: Nc1c(NCCC2=CCCCC2)ncnc1-n1cccn1] | 284.4 | C15H20N6 |
| 12 | 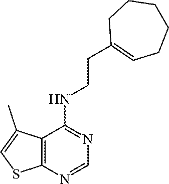 [SMILES: Cc1csc2ncnc(NCCC3=CCCCCC3)c12] | 287.4 | C16H21N3S |
| 13 | 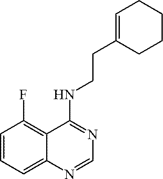 [SMILES: Fc1cccc2ncnc(NCCC3=CCCCC3)c12] | 271.3 | C16H18N3F |
| 14 | 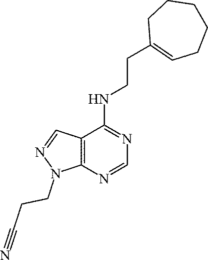 [SMILES: N#CCCn1ncc2c(NCCC3=CCCCCC3)ncnc21] | 310.4 | C17H22N6 |
| 15 | 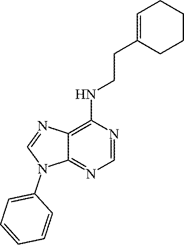 [SMILES: C1=C(CCNc2ncnc3c2ncn3-c2ccccc2)CCCC1] | 319.4 | C19H21N5 |
| 16 | 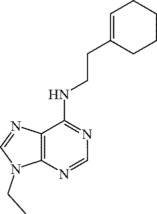 [SMILES: CCn1cnc2c(NCCC3=CCCCC3)ncnc21] | 271.4 | C15H21N5 |
| 17 | 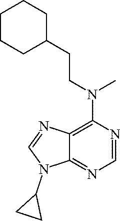 [SMILES: CN(CCC1CCCCC1)c1ncnc2c1ncn2C1CC1] | 299.4 | C17H25N5 |
| 18 | 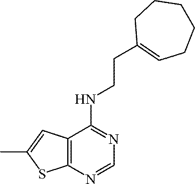 [SMILES: Cc1cc2c(NCCC3=CCCCCC3)ncnc2s1] | 287.4 | C16H21N3S |
| 19 | 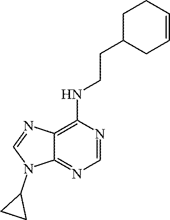 [SMILES: C1=CCC(CCNc2ncnc3c2ncn3C2CC2)CC1] | 283.4 | C16H21N5 |
| 20 | 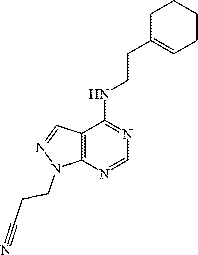 [SMILES: N#CCCn1ncc2c(NCCC3=CCCCC3)ncnc21] | 296.4 | C16H20N6 |
| 21 | 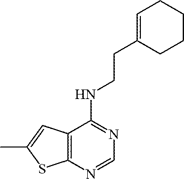 [SMILES: Cc1cc2c(NCCC3=CCCCC3)ncnc2s1] | 273.4 | C15H19N3S |
| 22 | 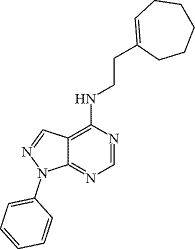 [SMILES: C1=C(CCNc2ncnc3c2cnn3-c2ccccc2)CCCCC1] | 333.4 | C20H23N5 |
| 23 | 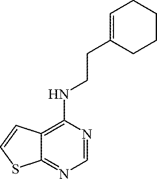 [SMILES: C1=C(CCNc2ncnc3sccc23)CCCC1] | 259.4 | C14H17N3S |
| 24 | 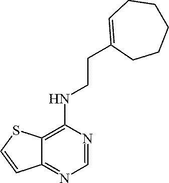 [SMILES: C1=C(CCNc2ncnc3ccsc23)CCCCC1] | 273.4 | C15H19N3S |
| 25 | 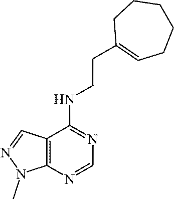 [SMILES: Cn1ncc2c(NCCC3=CCCCCC3)ncnc21] | 271.4 | C15H21N5 |
| 26 | 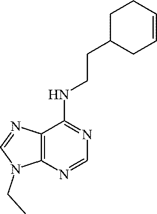 [SMILES: CCn1cnc2c(NCCC3CC=CCC3)ncnc21] | 271.4 | C15H21N5 |
| 27 | 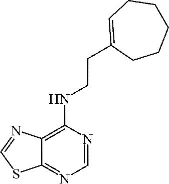 [SMILES: C1=C(CCNc2ncnc3scnc23)CCCCC1] | 274.4 | C14H18N4S |
| 28 | 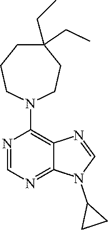 [SMILES: CCC1(CC)CCCN(c2ncnc3c2ncn3C2CC2)CC1] | 313.4 | C18H27N5 |
| 29 | 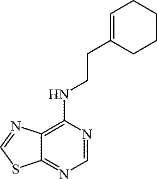 [SMILES: C1=C(CCNc2ncnc3scnc23)CCCC1] | 260.4 | C13H16N4S |
| 30 | 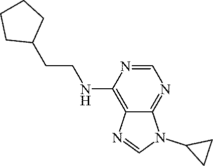 [SMILES: c1nc(NCCC2CCCC2)c2ncn(C3CC3)c2n1] | 271.4 | C15H21N5 |
| 31 | 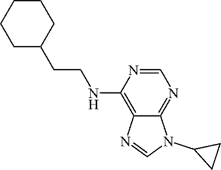 [SMILES: c1nc(NCCC2CCCCC2)c2ncn(C3CC3)c2n1] | 285.4 | C16H23N5 |
| 32 | 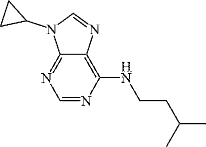 [SMILES: CC(C)CCNc1ncnc2c1ncn2C1CC1] | 245.3 | C13H19N5 |
| 33 | 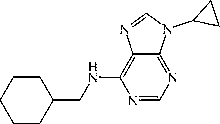 [SMILES: c1nc(NCC2CCCCC2)c2ncn(C3CC3)c2n1] | 271.4 | C15H21N5 |
| 34 | 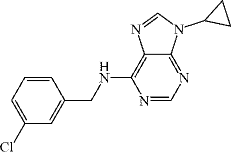 [SMILES: Clc1cccc(CNc2ncnc3c2ncn3C2CC2)c1] | 299.8 | C15H14N5Cl |
| 35 | 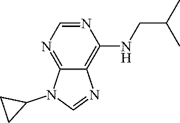 [SMILES: CC(C)CNc1ncnc2c1ncn2C1CC1] | 231.3 | C12H17N5 |
| 36 | 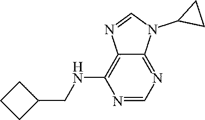 [SMILES: c1nc(NCC2CCC2)c2ncn(C3CC3)c2n1] | 243.3 | C13H17N5 |
| 37 | 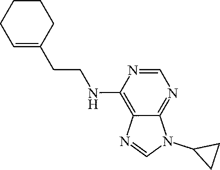 [SMILES: C1=C(CCNc2ncnc3c2ncn3C2CC2)CCCC1] | 283.4 | C16H21N5 |
| 38 | 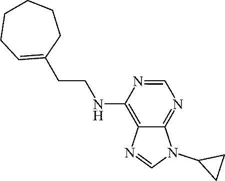 [SMILES: C1=C(CCNc2ncnc3c2ncn3C2CC2)CCCCC1] | 297.4 | C17H23N5 |
| 39 | 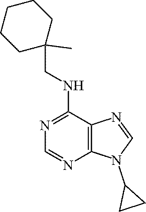 [SMILES: CC1(CNc2ncnc3c2ncn3C2CC2)CCCCC1] | 285.4 | C16H23N5 |
| 40 | 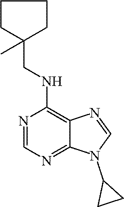 [SMILES: CC1(CNc2ncnc3c2ncn3C2CC2)CCCC1] | 271.4 | C15H21N5 |
| 41 | 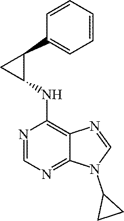 [SMILES: c1ccc([C@@H]2C[C@H]2Nc2ncnc3c2ncn3C2CC2)cc1] | 291.4 | C17H17N5 |
| 42 | 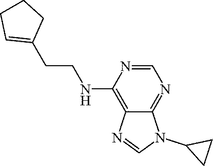 [SMILES: C1=C(CCNc2ncnc3c2ncn3C2CC2)CCC1] | 269.4 | C15H19N5 |
| 43 | 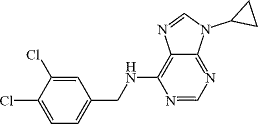 [SMILES: Clc1ccc(CNc2ncnc3c2ncn3C2CC2)cc1Cl] | 334.2 | C15H13N5C12 |
| 44 | 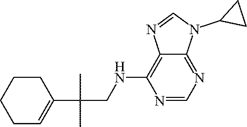 [SMILES: CC(C)(CNc1ncnc2c1ncn2C1CC1)C1=CCCCC1] | 311.4 | C18H25N5 |
| 45 | 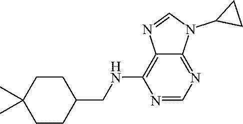 [SMILES: CC1(C)CCC(CNc2ncnc3c2ncn3C2CC2)CC1] | 299.4 | C17H25N5 |
| 46 | 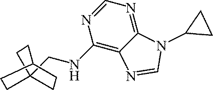 [SMILES: c1nc(NCC23CCC(CC2)CC3)c2ncn(C3CC3)c2n1] | 297.4 | C17H23N5 |
| 47 | 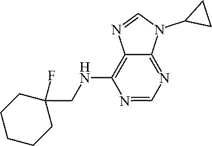 [SMILES: FC1(CNc2ncnc3c2ncn3C2CC2)CCCCC1] | 289.4 | C15H20N5F |
| 48 | 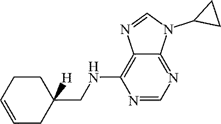 [SMILES: C1=CC[C@@H](CNc2ncnc3c2ncn3C2CC2)CC1] | 269.4 | C15H19N5 |
| 49 | 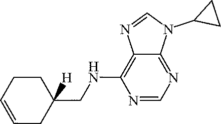 [SMILES: C1=CC[C@@H](CNc2ncnc3c2ncn3C2CC2)CC1] | 269.4 | C15H19N5 |

Furthermore, all compounds of the present disclosure which exist in free base or acid form can be converted to their pharmaceutically acceptable salts by treatment with the appropriate inorganic or organic base or acid by methods known to one skilled in the art. Salts of the compounds of the present disclosure can be converted to their free base or acid form by standard techniques.

Some embodiments are directed to a (pharmaceutical) composition comprising a pharmaceutically acceptable carrier or excipient and a compound of Formula I, II, or III.

Some embodiments are directed to a (pharmaceutical) medicament comprising a pharmaceutically acceptable carrier or excipient and a compound of Formula I, II, or III, or composition comprising the same.

Some embodiments are directed to a composition or medicament including a compound of Formula I, II, or III for use in (i) increasing klotho gene expression, (ii) increasing circulating and/or soluble Klotho protein levels, preferably through increasing klotho gene expression, (iii) treating Klotho protein deficiency, and/or (iv) treating other related conditions or affecting other related molecular mechanisms, in a mammal or mammalian subject (in need thereof). In some embodiments, the mammal or mammalian subject is human.

Some embodiments are directed to use of the compound of Formula I, II, or III, a pharmaceutical compositions comprising the same, or a medicament comprising the same, for (i) increasing klotho gene expression, (ii) increasing circulating and/or soluble Klotho protein levels, preferably through increasing klotho gene expression, (iii) treating Klotho protein deficiency, and/or (iv) treating other related conditions or affecting other related molecular mechanisms, in a mammal or mammalian subject (in need thereof). In some embodiments, the mammal or mammalian subject is human.

Some embodiments are directed to a method of (i) increasing klotho gene expression, (ii) increasing circulating and/or soluble Klotho protein levels, preferably through increasing klotho gene expression, (iii) treating Klotho protein deficiency, and/or (iv) treating other related conditions or affecting other related molecular mechanisms, in a mammal or mammalian subject (in need thereof), the method comprising administering a compound according to Formula I, II, or III, a pharmaceutical compositions comprising the same, or a medicament comprising the same, to the mammal or mammalian subject (in need thereof). In some embodiments, the mammal or mammalian subject is human.

Embodiments of the present disclosure are designed to be effective for increasing klotho gene expression in (mammalian) patients to which the novel compound(s), or composition(s) comprising the same, is/are administered. An increase in klotho gene expression can lead directly to an increase in circulating and/or soluble Klotho protein level(s). Those skilled in the art will appreciate that any disease or condition (particularly in older patients) that is caused, worsened, or exacerbated, in whole or in part, by (or associated with) low or diminished Klotho protein levels, may be addressed and/or treated (post-diagnosis or prophylactically) by administration of the novel compound(s), or composition(s) comprising the same, disclosed herein.

Some embodiments may include any of the features, options, and/or possibilities set out elsewhere in the present disclosure, including in other aspects or embodiments of the present disclosure. It is also noted that each of the foregoing, following, and/or other features described herein represent a distinct embodiment of the present disclosure. Moreover, combinations of any two or more of such features represent distinct embodiments of the present disclosure. Such features or embodiments can also be combined in any suitable combination and/or order without departing from the scope of this disclosure. Thus, each of the features described herein can be combinable with any one or more other features described herein in any suitable combination and/or order. Accordingly, the present disclosure is not limited to the specific combinations of exemplary embodiments described in detail herein.

Additional features and advantages of exemplary embodiments of the present disclosure will be set forth in the description that follows, and in part will be obvious from the description, or may be learned by the practice of such exemplary embodiments. The features and advantages of such embodiments may be realized and obtained by means of the instruments and combinations particularly pointed out in the appended claims. These and other features will become more fully apparent from the following description and appended claims, or may be learned by the practice of such exemplary embodiments as set forth hereinafter.

### BRIEF DESCRIPTION OF THE DRAWINGS

In order to describe the manner in which the above-recited and other advantages and features of the present disclosure can be obtained, a more particular description of the embodiments briefly described above will be rendered by reference to specific embodiments thereof which are illustrated in the appended drawings. For better understanding, the like elements have been designated by like reference numbers throughout the figure(s). Understanding that these drawings depict only typical embodiments of the disclosure and are not therefore to be considered to be limiting of its scope, the disclosure will be described and explained with additional specificity and detail through the use of the accompanying drawing(s) in which:

FIG. 1 depicts 49 illustrative compounds and information associated with the same.

FIG. 2 illustrates change in systolic blood pressure over time in rats treated with illustrative Compound 49.

FIG. 3 illustrates change in diastolic blood pressure over time in rats treated with illustrative Compound 49.

FIG. 4A illustrates systolic blood pressure in male mice treated with illustrative Compound 49.

FIG. 4B illustrates diastolic blood pressure in male mice treated with illustrative Compound 49.

FIG. 4C illustrates mean blood pressure in male mice treated with illustrative Compound 49.

FIG. 4D illustrates grip strength in male mice treated with illustrative Compound 49.

FIG. 5A illustrates systolic blood pressure in female mice treated with illustrative Compound 49.

FIG. 5B illustrates diastolic blood pressure in female mice treated with illustrative Compound 49.

FIG. 5C illustrates mean blood pressure in female mice treated with illustrative Compound 49.

FIG. 5D illustrates grip strength in female mice treated with illustrative Compound 49.

### DETAILED DESCRIPTION

Before describing various embodiments of the present disclosure in detail, it is to be understood that this disclosure is not limited only to the specific parameters, verbiage, and description of the particularly exemplified systems, methods, and/or products that may vary from one embodiment to the next. Thus, while certain embodiments of the present disclosure will be described in detail, with reference to specific features (e.g., configurations, parameters, properties, steps, components, ingredients, members, elements, parts, and/or portions, etc.), the descriptions are illustrative and are not to be construed as limiting the scope of the present disclosure and/or the claimed invention. In addition, the terminology used herein is for the purpose of describing the embodiments, and is not necessarily intended to limit the scope of the present disclosure and/or the claimed invention.

Unless defined otherwise, all technical and scientific terms used herein have the same meaning as commonly understood by one of ordinary skill in the art to which the present disclosure pertains.

Various aspects of the present disclosure, including systems, methods, and/or products may be illustrated with reference to one or more embodiments, which are exemplary in nature. As used herein, the terms “embodiment” mean “serving as an example, instance, or illustration,” and should not necessarily be construed as preferred or advantageous over other aspects disclosed herein. In addition, reference to an “embodiment” of the present disclosure or invention is intended to provide an illustrative example without limiting the scope of the invention, which is indicated by the appended claims.

As used in this specification and the appended claims, the singular forms “a,” “an” and “the” each contemplate, include, and specifically disclose both the singular and plural referents, unless the context clearly dictates otherwise. For example, reference to a “protein” contemplates and specifically discloses one, as well as a plurality of (e.g., two or more, three or more, etc.) proteins. Similarly, use of a plural referent does not necessarily require a plurality of such referents, but contemplates, includes, specifically discloses, and/or provides support for a single, as well as a plurality of such referents, unless the context clearly dictates otherwise.

As used throughout this disclosure, the words “can” and “may” are used in a permissive sense (i.e., meaning having the potential to), rather than the mandatory sense (i.e., meaning must). Additionally, the terms “including,” “having,” “involving,” “containing,” “characterized by,” variants thereof (e.g., “includes,” “has,” and “involves,” “contains,” etc.), and similar terms as used herein, including the claims, shall be inclusive and/or open-ended, shall have the same meaning as the word “comprising” and variants thereof (e.g., “comprise” and “comprises”), and do not exclude additional, un-recited elements or method steps, illustratively.

The term “condition” refers to any disorder, disease, injury, or illness, as understood by those skilled in the art, that is manifested or anticipated in a patient. Manifestation of such a condition can be an early, middle, or late stage manifestation, as known in the art, including pre-condition symptoms, signs, or markers. Anticipation of such a condition can be or include the predicted, expected, envisioned, presumed, supposed, and/or speculated occurrence of the same, whether founded in scientific or medical evidence, risk assessment, or mere apprehension or trepidation.

The term “patient,” as used herein, is synonymous with the term “subject” and generally refers to any animal under the care of a medical professional, as that term is defined herein, with particular reference to (i) humans (under the care of a doctor, nurse, or medical assistant or volunteer) and (ii) non-human animals, such as non-human mammals (under the care of a veterinarian or other veterinary professional, assistant, or volunteer).

Embodiments of the present disclosure are also meant to encompass all pharmaceutically acceptable compounds according to Formula I that are isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹p, ³²p, ³⁵S, ¹⁸F, ³⁶CI, ¹²³I, and ¹²⁵I, respectively. These radiolabeled compounds may be useful to help determine or measure the effectiveness of the compounds, by characterizing, for example, the site or mode of action, or binding affinity to pharmacologically important site of action. Certain isotopically-labelled compounds according to Formula I, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formula I can generally be prepared by conventional techniques known to those skilled in the art or by processes analogous to those described in the Preparations and Examples as set out below using an appropriate isotopically-labeled reagent in place of the non-labeled reagent previously employed.

Embodiments of the present disclosure may also encompass the in vivo metabolic products of the disclosed compounds. Such products may result from, for example, the oxidation, reduction, hydrolysis, amidation, esterification, and the like of the administered compound, primarily due to enzymatic processes. Accordingly, embodiments of the present disclosure include compounds produced by a process comprising administering a compound of this present disclosure to a mammal for a period of time sufficient to yield a metabolic product thereof. Such products are typically identified by administering a radiolabeled compound of the present disclosure in a detectable dose to an animal, such as rat, mouse, guinea pig, monkey, or to human, allowing sufficient time for metabolism to occur, and isolating its conversion products from the urine, blood or other biological samples.

The term(s) “carrier, diluent and/or excipient,” as well as “pharmaceutically acceptable carrier, diluent and/or excipient” include, without limitation, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The term “salt” or “pharmaceutically acceptable salt” includes both acid and base addition salts.

Salts may include “acid addition salt” or “pharmaceutically acceptable acid addition salt”, which refer to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

Salts may include “base addition salt” or “pharmaceutically acceptable base addition salt”, which refer to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallizations produce a solvate of the compound of the present disclosure. As used herein, the term “solvate” refers to an aggregate that comprises one or more molecules of a compound of the present disclosure with one or more molecules of solvent. The solvent may be water, in which case the solvate may be a hydrate. Alternatively, the solvent may be an organic solvent. Thus, embodiments of the compounds of the present disclosure may exist as a hydrate, including a monohydrate, dihydrate, hemihydrate, sesquihydrate, trihydrate, tetrahydrate and the like, as well as the corresponding solvated forms. Embodiments of the compound of the present disclosure may be true solvates, while in other cases, the compound of the present disclosure may merely retain adventitious water or be a mixture of water plus some adventitious solvent.

A “pharmaceutical composition” refers to a formulation of a compound of the present disclosure and a medium generally accepted in the art for the delivery of the biologically active compound to mammals, e.g., humans. Such a medium includes all pharmaceutically acceptable carriers, diluents or excipients therefor.

“Mammal” includes humans and both domestic animals such as laboratory animals and household pets (e.g., cats, dogs, swine, cattle, sheep, goats, horses, rabbits), and non-domestic animals such as wildlife and the like.

“Effective amount” or “therapeutically effective amount” refers to that amount of a compound of the present disclosure which, when administered to a mammal, preferably a human, is sufficient to effect treatment, as defined below, of a disease associated with overexpression of a cyclin-dependent kinase (CDK) in the mammal, preferably a human. The amount of a compound of the present disclosure which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest in a mammal, preferably a human, having the disease or condition of interest, and includes:
(i) preventing the disease or condition from occurring in a mammal, in particular, when such mammal is predisposed to the condition but has not yet been diagnosed as having it;
(ii) inhibiting the disease or condition, i.e., arresting its development;
(iii) relieving the disease or condition, i.e., causing regression of the disease or condition; or
(iv) relieving the symptoms resulting from the disease or condition, i.e., relieving pain without addressing the underlying disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

Compounds of the present disclosure, or their pharmaceutically acceptable salts may contain one or more asymmetric centers and may thus give rise to enantiomers, diastereomers, and other stereoisomeric forms that may be defined, in terms of absolute stereochemistry, as (R)- or (S)— or, as (D)- or (L)- for amino acids. The present disclosure is meant to include all such possible isomers, as well as their racemic and optically pure forms. Optically active (+) and (-), (R)— and (S)—, or (D)- and (L)- isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. Embodiments of the present disclosure contemplate various stereoisomers and mixtures thereof and includes “enantiomers”, which refers to two stereoisomers whose molecules are nonsuperimposeable mirror images of one another.

A “tautomer” refers to a proton shift from one atom of a molecule to another atom of the same molecule. Embodiments of the present disclosure include tautomers of any said compounds.

For the sake of brevity, the present disclosure may recite a list or range of numerical values. It will be appreciated, however, that where such a list or range of numerical values (e.g., greater than, less than, up to, at least, and/or about a certain value, and/or between two recited values) is disclosed or recited, any specific value or range of values falling within the disclosed values or list or range of values is likewise specifically disclosed and contemplated herein.

To facilitate understanding, like references (i.e., like naming of components and/or elements) have been used, where possible, to designate like elements common to different embodiments of the present disclosure. Similarly, like components, or components with like functions, will be provided with similar reference designations, where possible. Specific language will be used herein to describe the exemplary embodiments. Nevertheless it will be understood that no limitation of the scope of the disclosure is thereby intended. Rather, it is to be understood that the language used to describe the exemplary embodiments is illustrative only and is not to be construed as limiting the scope of the disclosure (unless such language is expressly described herein as essential).

While the detailed description is separated into sections, the section headers and contents within each section are for organizational purposes only and are not intended to be self-contained descriptions and embodiments or to limit the scope of the description or the claims. Rather, the contents of each section within the detailed description are intended to be read and understood as a collective whole, where elements of one section may pertain to and/or inform other sections. Accordingly, embodiments specifically disclosed within one section may also relate to and/or serve as additional and/or alternative embodiments in another section having the same and/or similar products, methods, and/or terminology.

### Compounds

Illustrative embodiments of the present disclosure include a compound according to Formula I:

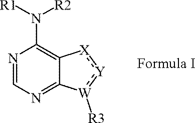

or a stereoisomer, tautomer or pharmaceutically acceptable salt thereof.

In various embodiments, X is (selected from (the group consisting of)) N, S, or C—R₄, wherein R₄ is (selected from (the group consisting of)) H or C1-C5 alkyl. In some embodiments, X is N. In some embodiments, X is S. In some embodiments, X is CH. In some embodiments, R₄ is H. In some embodiments, R₄ is C1-C5 alkyl.

In various embodiments, Y is (selected from (the group consisting of)) N or C—R₄, wherein R₄ is (selected from (the group consisting of)) H or C1-C5 alkyl. In some embodiments, Y is N. In some embodiments, Y is CH. In some embodiments, R₄ is H. In some embodiments, R₄ is C1-C5 alkyl.

In various embodiments, the bond between X-Y is a single bond or a double bond. In some embodiments, the bond between X-Y is a single bond. In some embodiments, the bond between X-Y is a double bond.

In various embodiments, W is (selected from (the group consisting of)) N, S, or C. In some embodiments, W is N. In some embodiments, W is S. In some embodiments, W is C.

In various embodiments, the bond between Y-W is a single bond or a double bond. In some embodiments, the bond between Y-W is a single bond. In some embodiments, the bond between Y-W is a double bond.

In various embodiments, R₁ is (selected from (the group consisting of)) H, CH₃, or, together with R₂, forms substituted or unsubstituted heterocyclic amine. In some embodiments, R₁ is H. In some embodiments, R₁ is CH₃. In various embodiments, R₁, together with R₂, forms substituted or unsubstituted heterocyclic amine.

In various embodiments, R₂ is (selected from (the group consisting of)):

(CH₂)_{Z}—(CR₅R₆)v—R₇, wherein Z is an integer from 0-2, V is an integer from 0-2, R₅ is (selected from (the group consisting of)) H or CH₃, R₆ is (selected from (the group consisting of)) H or CH₃; and R₇ is (selected from (the group consisting of)):
(i) saturated or unsaturated C3-C8 substituted or unsubstituted cycloalkyl or bicycloalkyl (e.g., bicyclo octane, preferably, bicyclo(2.2.2)octane), optionally substituted at one or more (ring) positions and each (ring) substituent is (selected from (the group consisting of)) halo, aryl (phenyl or benzyl), or branched or unbranched C1-C3 alkyl;
(ii) substituted or unsubstituted aryl (phenyl or benzyl), optionally substituted at one or more (1 or 2) (ring) positions and each (ring) substituent is (selected from (the group consisting of))halo or branched or unbranched C1-C3 alkyl; and/or
(iii) branched or unbranched C1-C3 alkyl (e.g., isopropyl);

C3-C7 substituted or unsubstituted cycloalkyl (e.g., 2-phenylcyclopropyl), optionally substituted at one or more (ring) positions and each (ring) substituent is (selected from (the group consisting of)) substituted or unsubstituted aryl (phenyl or benzyl), branched or unbranched C1-C3 substituted or unsubstituted alkyl; and/or

together with R₁, forms substituted or unsubstituted heterocyclic amine.

In some embodiments, R₂ is not C3 cycloalkyl, preferably substituted with aryl (or phenyl or benzyl), preferably 2-phenylcyclopropyl. In some embodiments, R₂ is not aryl (or phenyl or benzyl), preferably substituted at one or more (ring) positions with halo, preferably chloro, more preferably 3-chlorobenzyl or 2,3-dichlorobenzyl. In some embodiments, R₂ is not:

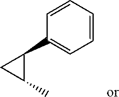

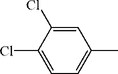

In various embodiments, R₃ is (selected from (the group consisting of)): nothing, H, alkyl, cycloalkyl, aryl (phenyl or benzyl), nitrile, (CH₂)_{Z}CN, wherein Z is an integer from 1-3, branched or unbranched C1-C3 substituted or unsubstituted alkyl, C3-C7 substituted or unsubstituted cycloalkyl, aryl (phenyl or benzyl) optionally substituted at one or more (ring) positions and each (ring) substituent is (selected from (the group consisting of)) branched or unbranched C1-C3 substituted or unsubstituted alkyl, halo, or nitrile. In some embodiments, R₃ is nothing. In some embodiments, R₃ is H. In some embodiments, R₃ is alkyl. In some embodiments, R₃ is cycloalkyl. In some embodiments, R₃ is aryl (phenyl or benzyl). In some embodiments, R₃ is nitrile. In some embodiments, R₃ is (CH₂)_{Z}CN, wherein Z is an integer from 1-3. In some embodiments, R₃ is branched or unbranched C1-C3 substituted or unsubstituted alkyl. In some embodiments, R₃ is C3-C7 substituted or unsubstituted cycloalkyl. In some embodiments, R₃ is aryl (phenyl or benzyl) substituted at one or more (ring) positions and each (ring) substituent is (selected from (the group consisting of)) branched or unbranched C1-C3 substituted or unsubstituted alkyl, halo, or nitrile. In some embodiments, R₃ is aryl (phenyl or benzyl) substituted at one or more (ring) positions with branched or unbranched C1-C3 substituted or unsubstituted alkyl, halo, or nitrile.

In some embodiments, R₁ is H and R₂ is (CH₂)_{Z}—(CR₅R₆)_{V}—R₇, wherein Z is an integer from 1-2, V is 0, and R₇ is (selected from (the group consisting of):
saturated or unsaturated C4-C7 cycloalkyl, optionally substitute at one or more (ring) positions with one or more methyl, preferably saturated C4-C6 cycloalkyl, optionally substitute at one or more (ring) positions with one or more methyl, more preferably unsubstituted saturated C4-C6 cycloalkyl or saturated C6 cycloalkyl optionally substituted at one or more (ring) position with one or more methyl or fluoro, still more preferably monounsaturated C5-C7 cycloalkyl, preferably 2-(1-cycloalkenyl or 4-(1-cycloalkenyl, more preferably 2-(1-cyclopentenyl), 2-(1-cyclohexenyl), 2-(1-cycloheptenyl), 4-(1-cyclopentenyl), 4-(1-cyclohexenyl), or 4-(1-cycloheptenyl);
bicycloalkyl, preferably bicyclo octane, more preferably, bicyclo(2.2.2)octane;
aryl (or phenyl or benzyl), preferably substituted at one or more (ring) positions with halo, preferably chloro, more preferably 3-chlorobenzyl or 2,3-dichlorobenzyl;
isopropyl; and/or
C3 cycloalkyl, preferably substituted with aryl (or phenyl or benzyl), preferably 2-phenylcyclopropyl.

In some embodiments, R1 is H and R2 is (CH₂)_{Z}—(CR₅R₆)_{V}—R₇, wherein Z is 1, V is 1, R₅ is CH₃, R₆ is CH₃, and R₇ is monounsaturated cyclohexyl, preferably 2-(1-cyclohexenyl).

In some embodiments, R₁ is CH₃ and R₂ is (CH₂)_{Z}—(CR₅R₆)_{V}—R₇, Z is 2, V is 0, and R₇ is cyclohexyl.

In some embodiments, R₁ together with R₂, forms substituted or unsubstituted heterocyclic amine, preferably, substituted azepane, more preferably 4,4-diethylazepane. In some embodiments, R₁ and R₂ form a substituted or unsubstituted, saturated or unsaturated heterocyclic amine, preferably a substituted azepane, aziridine, azetidine, pyrrolidine, piperidine, or azocane, more preferably azepane, still more preferably 4,4-diethyl azepane.

In some embodiments:
X is N, Y is C—R₄, R₄ is H, W is N, the bond between X-Y is a double bond, and the bond between Y-W is a single bond, and wherein R₃ is preferably alkyl or cycloalkyl, more preferably ethyl or cyclopropyl;
X is C—R₄, Y is C—R₄, R₄ is H, W is N, the bond between X-Y is a double bond, and the bond between Y-W is a single bond, and preferably, wherein R₃ is H;
X is C—R₄, R₄ is H, Y is N, W is N, the bond between X-Y is a double bond, and the bond between Y-W is a single bond, and preferably, wherein R₃ is preferably (i) alkyl, more preferably methyl, (ii) substituted or unsubstituted aryl, phenyl or benzyl, still more preferably fluorophenyl or fluorobenzyl, or (iii) nitrile, preferably ethanenitrile or ethyl cyanide;
X is N or C—R₄, Y is C—R₄, each R₄ is, independently, H or CH₃, W is S, the bond between X-Y is a double bond, and the bond between Y-W is a single bond, and wherein R₃ is nothing;
X is C—R₄, Y is C—R₄, each R₄ is, independently, H or CH₃, W is S, the bond between X-Y is a double bond, and the bond between Y-W is a single bond, and wherein R₃ is nothing;
X is N, Y is C—R₄, R₄ is H, W is S, the bond between X-Y is a doble bond, and the bond between Y-W is a single bond, and wherein R₃ is nothing;
X is N, Y is C—R₄, R₄ is H, W is N, the bond between X-Y is a double bond, and the bond between Y-W is a single bond, and wherein R₃ is preferably C2-C3 alkyl or cycloalkyl, preferably, ethyl or cyclopropyl; or
X is S, Y is C—R₄, R₄ is H, W is C, the bond between X-Y is a single bond, and the bond between Y-W is a double bond, and wherein R₃ is preferably H.

In some embodiments, X is N, Y is C—R₄, R₄ is H, W is N, the bond between X-Y is a double bond, and the bond between Y-W is a single bond, and wherein R₃ is preferably alkyl or cycloalkyl, more preferably ethyl or cyclopropyl.

In some embodiments, X is C—R₄, Y is C—R₄, R₄ is H, W is N, the bond between X-Y is a double bond, and the bond between Y-W is a single bond, and preferably, wherein R₃ is H.

In some embodiments, X is C—R₄, R₄ is H, Y is N, W is N, the bond between X-Y is a double bond, and the bond between Y-W is a single bond, and preferably, wherein R₃ is preferably (selected from (the group consisting of)) (i) alkyl, more preferably methyl, (ii) substituted or unsubstituted aryl, phenyl or benzyl, still more preferably fluorophenyl or fluorobenzyl, or (iii) nitrile, preferably ethanenitrile or ethyl cyanide.

In some embodiments, X is N or C—R₄, Y is C—R₄, each R₄ is, independently, H or CH₃, W is S, the bond between X-Y is a double bond, and the bond between Y-W is a single bond, and wherein R₃ is nothing.

In some embodiments, X is C—R₄, Y is C—R₄, each R₄ is, independently, H or CH₃, W is S, the bond between X-Y is a double bond, and the bond between Y-W is a single bond, and wherein R₃ is nothing.

In some embodiments, X is N, Y is C—R₄, R₄ is H, W is S, the bond between X-Y is a doble bond, and the bond between Y-W is a single bond, and wherein R₃ is nothing.

In some embodiments, X is N, Y is C—R₄, R₄ is H, W is N, the bond between X-Y is a double bond, and the bond between Y-W is a single bond, and wherein R₃ is preferably (selected from (the group consisting of)) C2-C3 alkyl or cycloalkyl, preferably, ethyl or cyclopropyl.

In some embodiments, X is S, Y is C—R₄, R₄ is H, W is C, the bond between X-Y is a single bond, and the bond between Y-W is a double bond, and wherein R₃ is preferably H.

In some embodiments, X is N, Y is C—R₄, R₄ is H, W is N, the bond between X-Y is a double bond, the bond between Y-W is a single bond, R₁ is H, R₂ is (CH₂)_{Z}—(CR₅R₆)_{V}—R₇, wherein Z is 1, V is 0, and R₇ is (selected from (the group consisting of)) unsubstituted monounsaturated C6 cycloalkyl or unsubstituted cyclohexenyl, preferably 4-(1-cyclohexenyl), more preferably R-4-(1-cyclohexenyl) or S-4-(1-cyclohexenyl), and R₃ is cyclopropyl.

In some embodiments, X is N, Y is C—R₄, R₄ is H, W is N, the bond between X-Y is a double bond, and the bond between Y-W is a single bond, R₃ is (selected from (the group consisting of)) alkyl or cycloalkyl, preferably ethyl or cyclopropyl, R₁ is H and R₂ is (CH₂)_{z}—(CR₅R₆)v—R₇, wherein Z is an integer from 1-2, V is 0, and R₇ is (selected from (the group consisting of)):
unsubstituted saturated C4-C6 cycloalkyl;
saturated C6 cycloalkyl substituted at one (ring) positions with fluoro, methyl, or dimethyl, preferably 1-fluorocyclohexyl, 1-methylcyclohexyl, or 4,4-dimethylcyclohexyl;
unsubstituted monounsaturated C5-C7 cycloalkyl, preferably 2-(1-cycloalkenyl or 4-(1-cycloalkenyl, more preferably 2-(1-cyclopentenyl), 2-(1-cyclohexenyl), 2-(1-cycloheptenyl), 4-(1-cyclopentenyl), 4-(1-cyclohexenyl), or 4-(1-cycloheptenyl);
bicyclo octane, more preferably, bicyclo(2.2.2)octane;
aryl (or phenyl or benzyl) substituted at one or two (ring) positions with halo, preferably chloro, more preferably 3-chlorobenzyl or 2,3-dichlorobenzyl;
isopropyl; and/or
C3 cycloalkyl substituted with aryl (or phenyl or benzyl), preferably 2-phenylcyclopropyl.

Illustrative embodiments of the present disclosure include a compound according to Formula Ia:

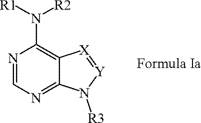

or a stereoisomer, tautomer or pharmaceutically acceptable salt thereof, wherein X, Y, R₁, R₂, and R₃ are as described for Formula I.

Illustrative embodiments of the present disclosure include a compound according to Formula II:

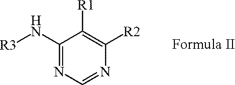

or a stereoisomer, tautomer or pharmaceutically acceptable salt thereof.

In various embodiments, R₁ is (selected from (the group consisting of) H or NH₂. In some embodiments, R₁ is H. In some embodiments, R₁ is NH₂.

In various embodiments, R₂ is (selected from (the group consisting of) saturated or unsaturated, heterocyclic amine or heterocyclic diamine. In some embodiments, R₂ is saturated or unsaturated heterocyclic amine. In some embodiments, R₂ is saturated or unsaturated heterocyclic diamine.

In some embodiments, R₂ is saturated heterocyclic amine, preferably N-pyrrolidine or N-piperidine, or unsaturated heterocyclic diamine, preferably N-pyrazole.

In some embodiments, when R₁ is H, then R₂ is N-pyrrolidine or N-piperidine.

In some embodiments, when R₁ is NH₂, then R₂ is N-pyrazole.

In various embodiments, R₃ is (CH₂)₂—R₇, wherein R₇ is unsaturated cycloalkyl.

In some embodiments, R₇ is monounsaturated C6-C7 cycloalkyl, preferably cyclohexenyl, more preferably 2-(1-cyclohexenyl), or cycloheptenyl, more preferably 2-(1-cycloheptenyl).

In some embodiments, the compound of Formula II is selected from the group consisting of Formulas IIa—IId—

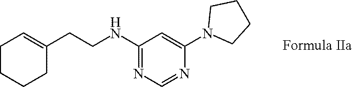 [SMILES: C1=C(CCNc2cc(N3CCCC3)ncn2)CCCC1]

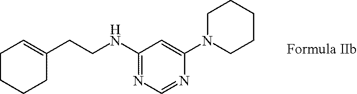 [SMILES: C1=C(CCNc2cc(N3CCCCC3)ncn2)CCCC1]

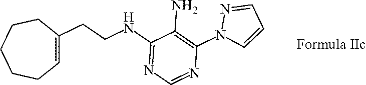 [SMILES: Nc1c(NCCC2=CCCCCC2)ncnc1-n1cccn1]

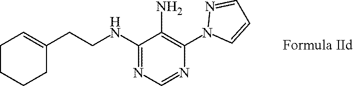 [SMILES: Nc1c(NCCC2=CCCCC2)ncnc1-n1cccn1]

or a stereoisomer, tautomer or pharmaceutically acceptable salt thereof.

Illustrative embodiments of the present disclosure include a compound according to Formula III:

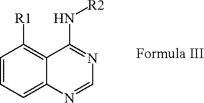

or a stereoisomer, tautomer or pharmaceutically acceptable salt thereof, wherein R₁ is halo; and R₂ is (CH₂)₂—R₇, wherein R₇ is unsaturated cycloalkyl. In some embodiments, R₁ is fluoro and R₇ is unsaturated cycloalkyl, preferably monounsaturated cycloalkyl, more preferably cyclohexenyl, more preferably 2-(1-cyclohexenyl), still most preferably, wherein the compound is according to Formula IIIa:

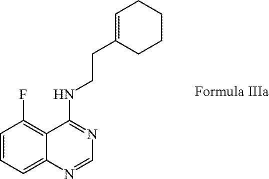 [SMILES: Fc1cccc2ncnc(NCCC3=CCCCC3)c12]

In some embodiments, the compound according to Formula I can be one of Compounds 1-49 of Table 1.

### Compositions and Medicaments

Administration of the compounds of the present disclosure, or their pharmaceutically acceptable salts, in pure form or in an appropriate pharmaceutical composition or medicament, can be carried out via any of the accepted modes of administration of agents for serving similar utilities. The pharmaceutical compositions or medicaments of embodiments of the present disclosure can be prepared by combining a compound of the present disclosure with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions or medicaments include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Pharmaceutical compositions or medicaments of the present disclosure are formulated so as to allow the active ingredients contained therein to be bioavailable upon administration of the composition or medicament to a patient. Compositions or medicaments that will be administered to a subject or patient take the form of one or more dosage units, where for example, a tablet may be a single dosage unit, and a container of a compound of the present disclosure in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition or medicament to be administered will, in any event, contain a therapeutically effective amount of a compound of the present disclosure, or a pharmaceutically acceptable salt thereof, for treatment of a disease or condition of interest in accordance with the teachings of this disclosure.

A pharmaceutical composition or medicament of some embodiments of the present disclosure may be in the form of a solid or liquid. In one aspect, the carrier(s) are particulate, so that the compositions or medicaments are, for example, in tablet or powder form. The carrier(s) may be liquid, with the compositions being, for example, an oral syrup, injectable liquid or an aerosol, which is useful in, for example, inhalation or inhalatory administration.

When intended for oral administration, the pharmaceutical composition or medicament is preferably in either solid or liquid form, where semi-solid, semi-liquid, suspension and gel forms are included within the forms considered herein as either solid or liquid.

As a solid composition or medicament for oral administration, the pharmaceutical composition or medicament may be formulated into a powder, granule, compressed tablet, pill, capsule, chewing gum, wafer or the like form. Such a solid composition or medicament will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition or medicament is in the form of a capsule, for example, a gelatin capsule, it may contain, in addition to materials of the above type, a liquid carrier such as polyethylene glycol or oil.

The pharmaceutical composition or medicament may be in the form of a liquid, for example, an elixir, syrup, solution, emulsion or suspension. The liquid may be for oral administration or for delivery by injection, as two examples. When intended for oral administration, preferred composition or medicament contain, in addition to the present compounds, one or more of a sweetening agent, preservatives, dye/colorant and flavor enhancer. In a composition or medicament intended to be administered by injection, one or more of a surfactant, preservative, wetting agent, dispersing agent, suspending agent, buffer, stabilizer and isotonic agent may be included.

The liquid pharmaceutical compositions and medicaments of some embodiments of the present disclosure, whether they be solutions, suspensions or other like form, may include one or more of the following adjuvants: sterile diluents such as water for injection, saline solution, preferably physiological saline, Ringer’s solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition or medicament is preferably sterile.

A liquid pharmaceutical composition or medicament of certain embodiments of the present disclosure intended for either parenteral or oral administration should contain an amount of a compound of the present disclosure such that a suitable dosage will be obtained.

In some embodiments, the pharmaceutical composition or medicament of the present disclosure may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition or medicament for topical administration. If intended for transdermal administration, the composition or medicament may include a transdermal patch or iontophoresis device.

The pharmaceutical composition or medicament of various embodiments of the present disclosure may be intended for rectal administration, in the form, for example, of a suppository, which will melt in the rectum and release the drug. The composition or medicament for rectal administration may contain an oleaginous base as a suitable nonirritating excipient. Such bases include, without limitation, lanolin, cocoa butter and polyethylene glycol.

Embodiments of the pharmaceutical composition or medicament of the present disclosure may include various materials, which modify the physical form of a solid or liquid dosage unit. For example, the composition or medicament may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition or medicament of some embodiments of the present disclosure in solid or liquid form may include an agent that binds to the compound of the present disclosure and thereby assists in the delivery of the compound. Suitable agents that may act in this capacity include a monoclonal or polyclonal antibody, a protein or a liposome.

The pharmaceutical composition or medicament of other embodiments of the present disclosure may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the present disclosure may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

In some embodiments, the pharmaceutical compositions or medicaments of the present disclosure may be prepared by methodology well known in the pharmaceutical art. For example, a pharmaceutical composition or medicament intended to be administered by injection can be prepared by combining a compound of the present disclosure with sterile, distilled water so as to form a solution. A surfactant may be added to facilitate the formation of a homogeneous solution or suspension. Surfactants are compounds that non-covalently interact with the compound of the present disclosure so as to facilitate dissolution or homogeneous suspension of the compound in the aqueous delivery system.

The compounds of the present disclosure, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy.

Compounds of the present disclosure, or pharmaceutically acceptable derivatives thereof, may also be administered simultaneously with, prior to, or after administration of one or more other therapeutic agents. Such combination therapy includes administration of a single pharmaceutical dosage formulation which contains a compound of the present disclosure and one or more additional active agents, as well as administration of the compound of the present disclosure and each active agent in its own separate pharmaceutical dosage formulation. For example, a compound of the present disclosure and the other active agent can be administered to the patient together in a single oral dosage composition such as a tablet or capsule, or each agent administered in separate oral dosage formulations. Where separate dosage formulations are used, the compounds of the present disclosure and one or more additional active agents can be administered at essentially the same time, i.e., concurrently, or at separately staggered times, i.e., sequentially; combination therapy is understood to include all these regimens.

In some embodiments, the concentration of the compound of Formula I provided in the pharmaceutical compositions or medicaments of the present disclosure is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001 %, w/w, w/v or v/v, of the pharmaceutical composition or medicament.

In some embodiments, the concentration of the compound of Formula I provided in the pharmaceutical compositions or medicaments of the present disclosure is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001%, w/w, w/v, or v/v, of the pharmaceutical composition or medicament.

In some embodiments, the concentration of the compound of the Formula I provided in the pharmaceutical compositions or medicaments of the present disclosure is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v, of the pharmaceutical composition or medicament.

In some embodiments, the concentration of the compound of Formula I provided in the pharmaceutical compositions or medicaments of the present disclosure is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v, of the pharmaceutical composition or medicament.

In some embodiments, the amount the compound of Formula I provided in the pharmaceutical compositions or medicaments of the present disclosure is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g of the pharmaceutical composition or medicament.

In some embodiments, the amount of the compound of Formula I provided in the pharmaceutical compositions or medicaments of the present disclosure is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g of the pharmaceutical composition or medicament.

In some embodiments, the amount of the compound of Formula I provided in the pharmaceutical compositions or medicaments of the present disclosure is in the range of 0.0001-10 g, 0.0005-9 g, 0.001-8 g, 0.005-7 g, 0.01-6 g, 0.05-5 g, 0.1-4 g, 0.5-4 g, or 1-3 g of the pharmaceutical composition or medicament.

### Chemical Synthesis

All chemicals, reagents and solvents were obtained from commercial vendors, such as Enamine, Sigma-Aldrich, and Fisher Scientific. Indicated reaction temperatures refer to those of the reaction bath, while room temperature (rt) is noted as 25° C. Analytical thin layer chromatography (TLC) was performed with glass backed silica plates (20× 20 cm, pH = 5, MF254). Visualization was accomplished using a 254 nm UV lamp. ¹H spectra were recorded on a 400 MHz spectrometer using solutions of samples in DMSO-d6 or other commercially-available deuterated solvents, as noted. Chemical shifts are reported in ppm with tetramethylsilane as standard. Data are reported as follows: chemical shift, number of protons, multiplicity (s = singlet, d = doublet, dd = doublet of doublet, t = triplet, q = quartet, b = broad, m = multiplet). All novel compounds were characterized by ¹H-NMR and mass spectroscopy (MS).

### Example 1. N-[(cyclohex-3-en-1-yl)methyl]-9-cyclopropyl-9H-purin-6-amine

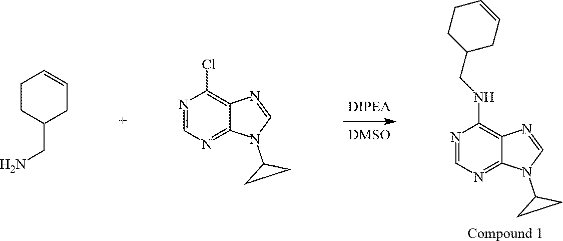

A stirred mixture of 1-(cyclohex-3-en-1-yl)methanamine (1.3 g, 11.7 mmol) and N,N-diisopropylethylamine (1.727 g, 13.4 mmol) in DMSO (15 mL) was stirred for 15 min at r.t. and 6-chloro-9-cyclopropyl-9H-purine (2.168 g, 11.1 mmol) was added. The reaction mixture was heated to 90° C. and stirred for 16 h. After cooling down, the resulting mixture was diluted with water and extracted with ethyl acetate. The organic layer was washed with water, dried over Na2SO4 and evaporated in vacuo at 50° C. to afford the crude residue, which was purified by flash column chromatography to obtain compound 1, N-[(cyclohex-3-en-1-yl)methyl]-9-cyclopropyl-9H-purin-6-amine (2.1 g, 70%). ¹H NMR (400 MHz, DMSO-d6): δ 8.18 (s, 1H), 8.08 (s, 1H), 7.77 (t, J = 10.2 Hz, 1H), 5.62 (s, 2H), 3.91 - 3.47 (m, 1H), 3.48 - 3.36 (m, 2H), 2.20 - 1.82 (m, 4H), 1.81 - 1.61 (m, 2H), 1.27 - 1.12 (m, 1H), 1.14 - 0.91 (m, 4H). MS: m/z 270 [M+H]+.

### Example 2. N-[2-(cyclohept-1-en-1-yl)ethyl]-7H-pyrrolo[2,3-d]pyrimidin-4-amine

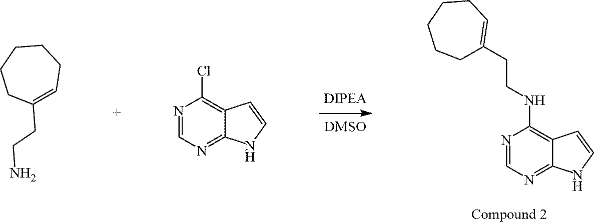

A stirred mixture of 2-(cyclohept-1-en-1-yl)ethan-1-amine (3.5 g, 12.7 mmol, hydrochloride salt) and N,N-diisopropylethylamine (3.328 g, 25.7 mmol) in DMSO (15 mL) was stirred for 15 min at r.t. and 4-chloro-7H-pyrrolo[2,3-d]pyrimidine (1.797 g, 11.7 mmol) was added. The reaction mixture was heated to 90° C. and stirred for 16 h. After coolingdown, the resulting mixture was diluted with water and extracted with ethyl acetate. The organic layer was washed with water, dried over Na2SO4 and evaporated in vacuo at 50° C. to afford the crude residue, which was purified by flash column chromatography to obtain compound 2, N-[2-(cyclohept-1-en-1-yl)ethyl]-7H-pyrrolo[2,3-d]pyrimidin-4-amine (1.238 g, 41%). ¹H NMR (400 MHz, DMSO-d6 δ 11.44 (s, 1H), 8.08 (s, 1H), 7.27 (t, J = 5.1 Hz, 1H), 7.03 (s, 1H), 6.50 (s, 1H), 5.58 (t, J = 6.3 Hz, 1H), 3.54 - 3.44 (m, 2H), 2.25 (t, J = 7.4 Hz, 2H), 2.18 - 2.08 (m, J = 10.4 Hz, 2H), 2.05 - 1.90 (m, J = 8.2 Hz, 2H), 1.75 - 1.57 (m, J = 5.4 Hz, 2H), 1.52 - 1.21 (m, 4H). MS: m/z 259 [M+H]+.

### Example 3. [00224] (R)-N-(cyclohex-3-en-1-ylmethyl)-9-cyclopropyl-9H-purin-6-amine (Compound 49)

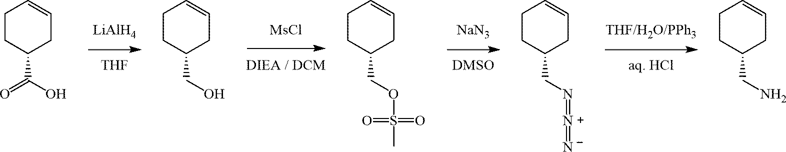

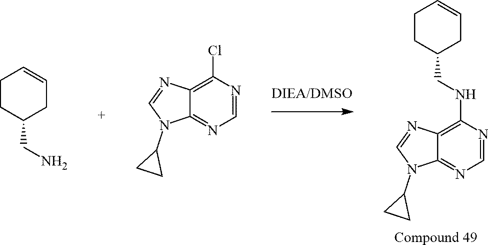

(R)-cyclohex-3-en-1-ylmethanol: A suspension of LiAlH₄ (35.52 g, 930 mmol) in anhydrous THF (1000 mL) was cooled to 0° C. Then compound (R)-cyclohex-3-ene-1-carboxylic acid (78.72 g, 620 mmol) was added dropwise at 0° C. under argon atmosphere. Upon completion, the reaction mixture was allowed to warm up to RT and stirred for 4 h. After that, H₂O and 5% aqueous NaOH (75 mL) were added and the mixture was stirred for 1 h. The suspension was filtrated; the filter cake was washed with THF. The combined filtrate was evaporated and distilled in the middle vacuo to obtain (R)-cyclohex-3-en-1-ylmethanol (55 g, 78% yield).

(R)-cyclohex-3-en-1-ylmethyl methanesulfonate: A solution of (R)-cyclohex-3-en-1-ylmethanol (54.84 g, 480 mmol) and DIEA (75.81 g, 580 mmol) in dichloromethane (1000 mL) was cooled to 0° C. followed by dropwise addition of MsCI (61.59 g, 540 mmol). Upon completion, the mixture was allowed to warm up to RT and stirred overnight. After the reaction was completed, the solution was diluted with water. The organic layer was washed with water, brine, dried over Na₂SO₄ and evaporated to obtain (R)-cyclohex-3-en-1-ylmethyl methanesulfonate (92.9 g, 99% yield).

(R)-4-(azidomethyl)cyclohex-1-ene: To a solution of (R)-cyclohex-3-en-1-ylmethyl methanesulfonate (92.92 g, 480 mmol) in DMSO (500 mL) NaN₃ (47.63 g, 730 mmol) was added. The mixture was stirred overnight at 50° C. After the reaction was completed, the mixture was poured into ice-cooled water and extracted with MTBE. The organic layer was washed with water, brine, dried over Na₂SO₄ and evaporated under reduced pressure to obtain (R)-4-(azidomethyl)cyclohex-1-ene (66.7 g, 99% yield).

(R)-cyclohex-3-en-1-ylmethanamine: A solution of (R)-4-(azidomethyl)cyclohex-1-ene (66.63 g, 480 mmol) in THF/H₂O 2:1 (1000 mL) was cooled to 0° C. Then PPh₃ (152.86 g, 580 mmol) was added portion-wise at 0° C. The mixture was allowed to warm up to RT and stirred overnight. After the reaction was completed, the mixture was cooled to 0° C. and 35% aqueous HCI was added dropwise to pH=3. The precipitate formed was collected and washed with H₂O. The filtrate was washed with MTBE. The aqueous layer was basified to pH=10 and extracted with MTBE. The organic layer was dried over Na₂SO₄ and evaporated under reduced pressure to obtain (R)-cyclohex-3-en-1-ylmethanamine (28 g, 52% yield).

(R)-N-(cyclohex-3-en-1-ylmethyl)-9-cyclopropyl-9H-purin-6-amine: A mixture of (R)-cyclohex-3-en-1-ylmethanamine (8.67 g, 80 mmol), 6-chloro-9-cyclopropyl-9H-purine (17.16 g, 70 mmol), and DIEA (24 g, 186 mmol) in DMSO (100 mL) was stirred at 90° C. for 16 h. Then an additional portion of compound 6 (5 g, 20 mmol) and DIEA (8 g, 60 mmol) were added and the mixture was stirred for 8 h at 90° C. After the reaction was completed, the solution was poured into ice-cooled water and extracted with MTBE. The organic layer was washed with water, brine, dried over Na₂SO₄ and filtered. The filtrate was treated with activated charcoal and evaporated under reduced pressure to obtain (R)-N-(cyclohex-3-en-1-ylmethyl)-9-cyclopropyl-9H-purin-6-amine (10.2 g, 51% yield). ¹H NMR (400 MHz, DMSO) δ 8.19 (s, 1H), 8.09 (s, 1H), 7.78 (s, 1H), 5.68 - 5.57 (m, 2H), 3.93 - 3.35 (m, 3H), 2.15 - 1.85 (m, 4H), 1.81 - 1.64 (m, 2H), 1.28 - 1.14 (m, 1H), 1.14 - 0.97 (m, 4H). MS: m/z 270 [M+H]+

### Example 4. Additional Illustrative Compounds

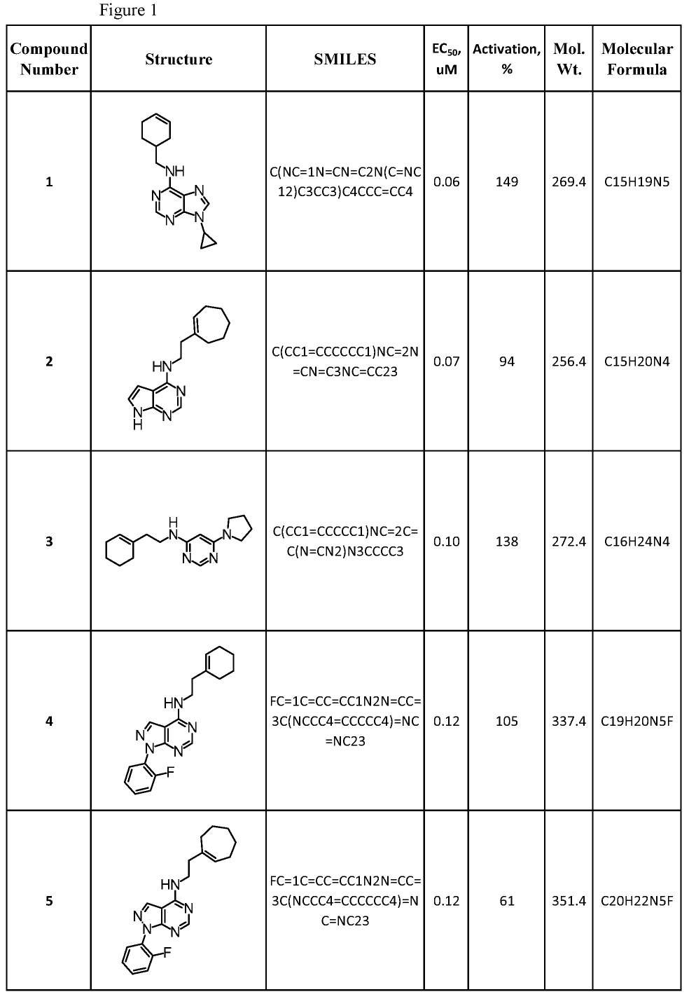

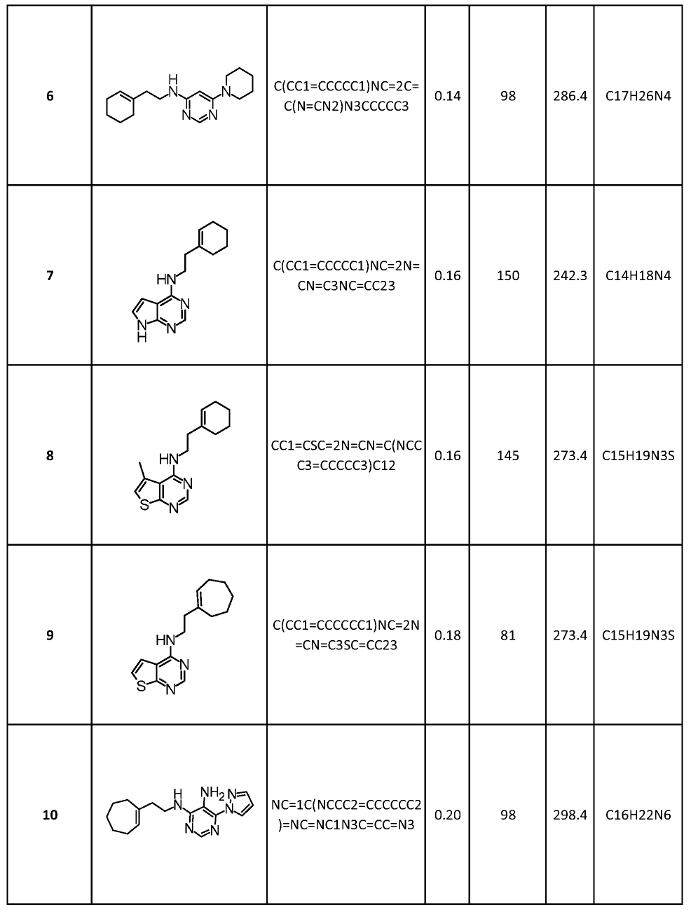

A listing of illustrative Compounds 1-49 are presented in FIG. 1. Compounds 3-48 are prepared and purified via similar methods to Examples 1, 2, and 3, with the appropriate amine and heterocyclic reagents, and are characterized by ¹H NMR, HPLC and MS.

### Assays and Experimentation

### Example 5. Table 2. Luciferase Reporter Assay to Evaluate Klotho Gene Expression Changes

HEK293 cells/pKlotho_Luc in suspension (20 µl/well, density 0.25×106 cells/ml) were dispensed into assay-ready 384-well tissue culture treated plates (plates (BD BioCoat Poly-D-Lys coated, Cat.354661) preloaded with 1000x stock of test compounds in DMSO (20 nL/well, 10 concentration points, 3x dilutions from 20 uM to 0.001 uM). Growth medium: DMEM, 1% FBS, Pen/Strep, 600 ug/ml G418. Negative control: DMSO. Positive control: 2 uM Compound H from King, et.al. Biochem. J. (2012) 441, 453-461. Cells were incubated in CO₂ incubator overnight. At the end of the incubation time, 20 ul/well of the luciferase assay solution (Steady-Luc Firefly HTS Assay Biotium, Cat 30028) were added to the plate and mixed on a VibraTranslator (Union Scientific). Plates were incubated for 5 min at 25° C. Steady glow luminescence signal was read using a BMG Labtech PHERAstar FSX reader.

TABLE 2

| Compound Number | EC₅₀, uM | Activation, % |
|---|---|---|
| 1 | 0.06 | 149 |
| 2 | 0.07 | 94 |
| 3 | 0.10 | 138 |
| 4 | 0.12 | 105 |
| 5 | 0.12 | 61 |
| 6 | 0.14 | 98 |
| 7 | 0.16 | 150 |
| 8 | 0.16 | 145 |
| 9 | 0.18 | 81 |
| 10 | 0.20 | 98 |
| 11 | 0.21 | 125 |
| 12 | 0.21 | 98 |
| 13 | 0.25 | 86 |
| 14 | 0.26 | 122 |
| 15 | 0.27 | 203 |
| 16 | 0.37 | 151 |
| 17 | 0.38 | 114 |
| 18 | 0.45 | 48 |
| 19 | 0.50 | 152 |
| 20 | 0.60 | 120 |
| 21 | 0.68 | 133 |
| 22 | 0.77 | 96 |
| 23 | 0.85 | 133 |
| 24 | 0.88 | 122 |
| 25 | 0.89 | 90 |
| 26 | 0.92 | 121 |
| 27 | 0.92 | 101 |
| 28 | 1.00 | 101 |
| 29 | 1.1 | 103 |
| 30 | 1.2 | 87 |
| 31 | 1.1 | 109 |
| 32 | 0.73 | 77 |
| 33 | 0.38 | 89 |
| 34 | 2.0 | 102 |
| 35 | 1.5 | 40 |
| 36 | 0.83 | 68 |
| 37 | 0.07 | 103 |
| 38 | 0.34 | 105 |
| 39 | 3.6 | 77 |
| 40 | 2.9 | 89 |
| 41 | >30 | N/A |
| 42 | 0.62 | 79 |
| 43 | >30 | N/A |
| 44 | 23.8 | 58 |
| 45 | 13.1 | 59 |
| 46 | 1.7 | 86 |
| 47 | 0.90 | 86 |
| 48 | 0.16 | 86 |
| 49 | 0.10 | 90 |

Accordingly, each of compounds 1-40, 42, and 44-49 were shown to be effective at increasing klotho gene expression.

### Aged Rat Efficacy Model

The aim of the study was to assess the efficacy of test compound in improving physiologic parameters in aged rats such as blood pressure values. The experiment included daily repeated administrations of the test compound for 14 consecutive days and follow up observations within a 15-day post-dosing period. Body weight (BWt) were monitored daily during the dosing period and twice a week during the post-dosing period. Blood pressure (BP) was measured on 0, 7th, 14th, 21^{st} and 28th day of the study. Study design, animal selection, handling and treatment were all in accordance with the CROs efficacy study protocols and Standard Operation Procedure, and the Animal Care Guidelines.

Fifteen aged male Wistar rats (22.5 months old; initial body weights ranged from 474.8 g to 672.4 g with an average body weight across the groups of 540.8 g) were randomized into two groups by body weight and systolic blood pressure: 11 animals distributed to compound 49-treated group and 4 animals - to vehicle-treated group. Two rats (rat# 8, rat# 16) previously exposed to local surgery due to tumor appearing, did not undergo to randomizing process and were placed one into compound-treated group (rat# 8) and another into vehicle-treated group (rat# 16). Rats in the compound-treated group were repeatedly PO dosed with Compound 49 at the dose of 50 mg/kg at the volume of 5 m1/kg with an interval of 24 h for thirteen consecutive days starting from Day 1 of the experimental period. Day 14 animals were treated with Compound 49 at ⅓ of the dose. Experimental animals from vehicle-treated group were repeatedly dosed with vehicle (0.75% PVP K30 and 0.025% sodium docusate in distilled water) at the volume of 5 ml/kg with an interval of 24 h for thirteen consecutive days starting from Day 1 of the experimental period and at Day 14 they obtained ⅓ of the vehicle volume.

Blood pressure was measured by the Tail-Cuff Method using the Coda Non-invasive Blood-Pressure System. Individual body weights and relative changes in body weight (compared to the appropriate weight on Day 0) were recorded. Experimental animals’ body weights were reduced during the administration period and gained during postdosing period in the same manner for Compound 49-treated and vehicle-treated groups.

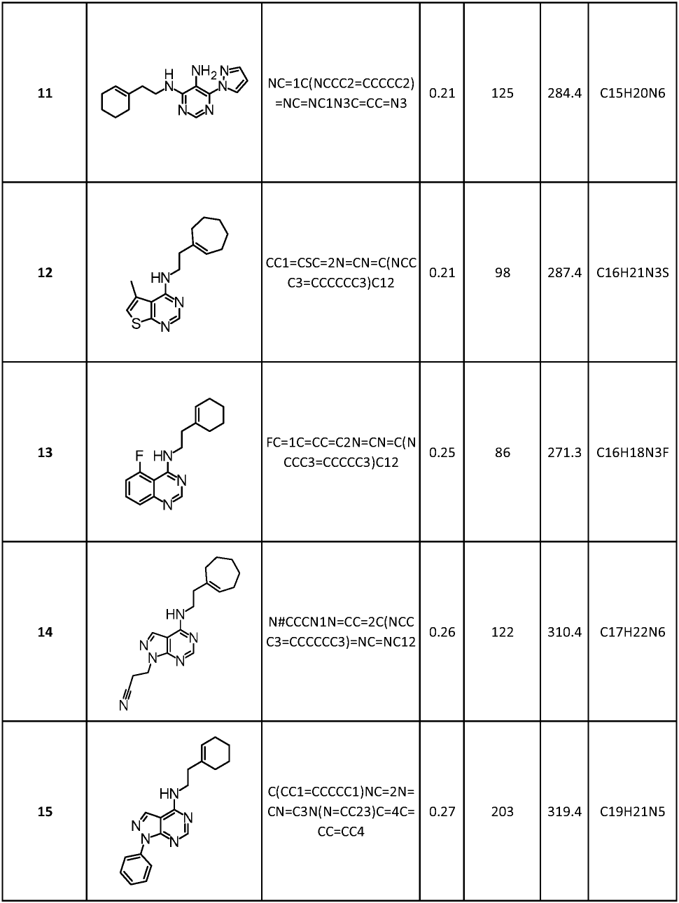

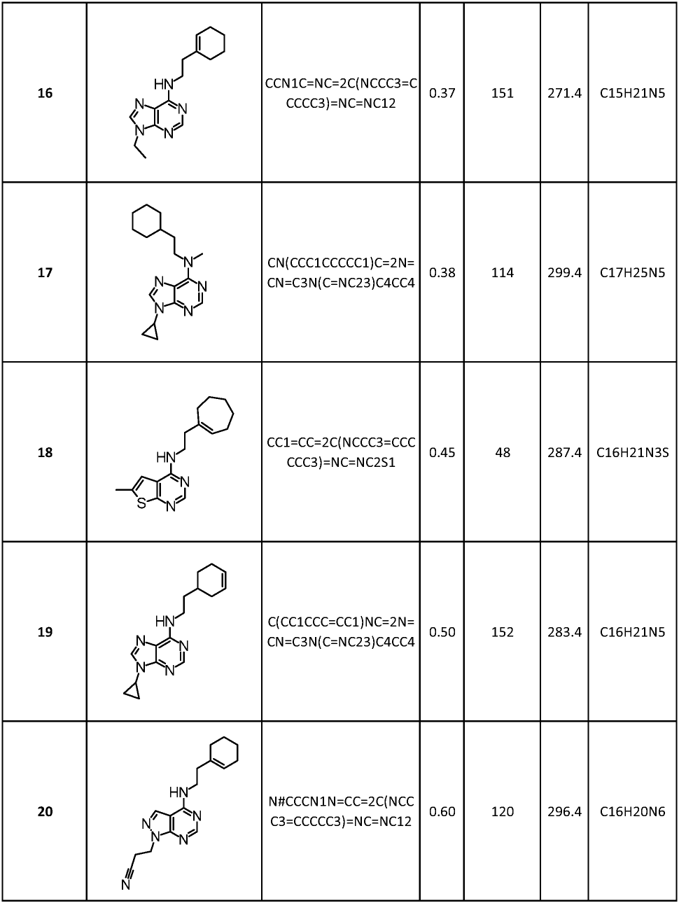

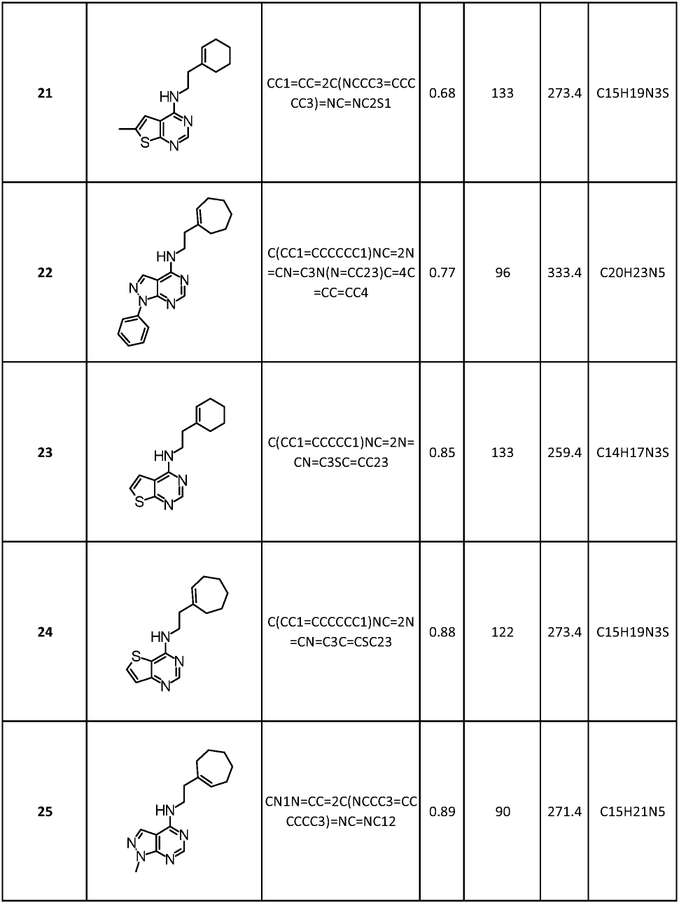

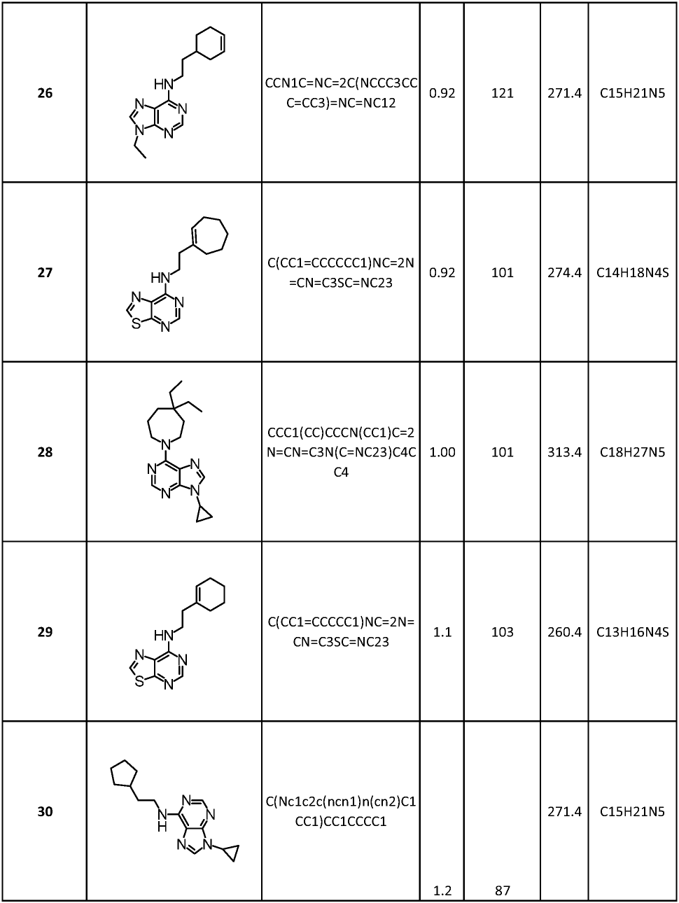

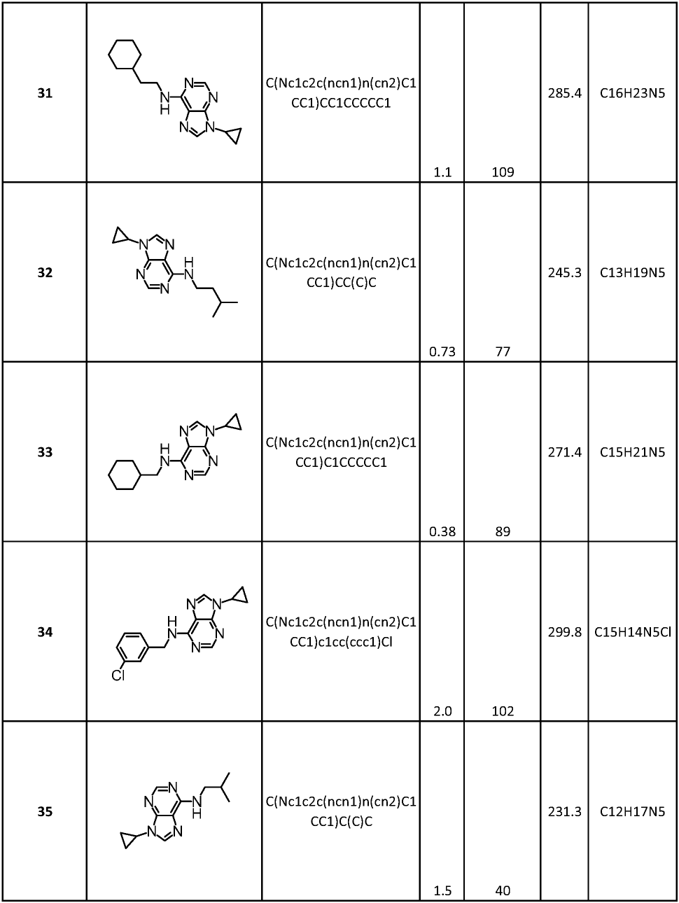

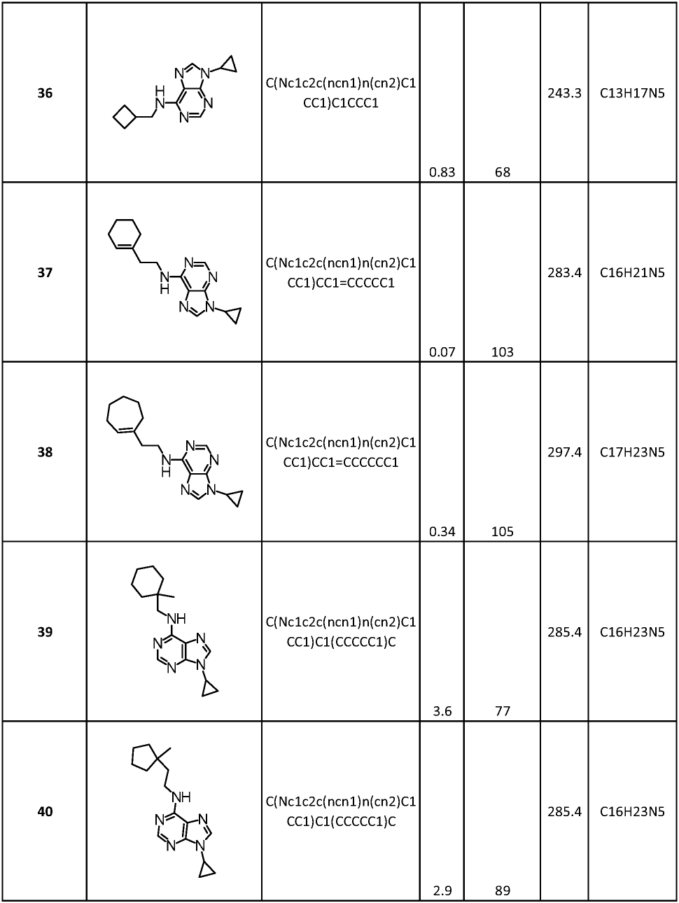

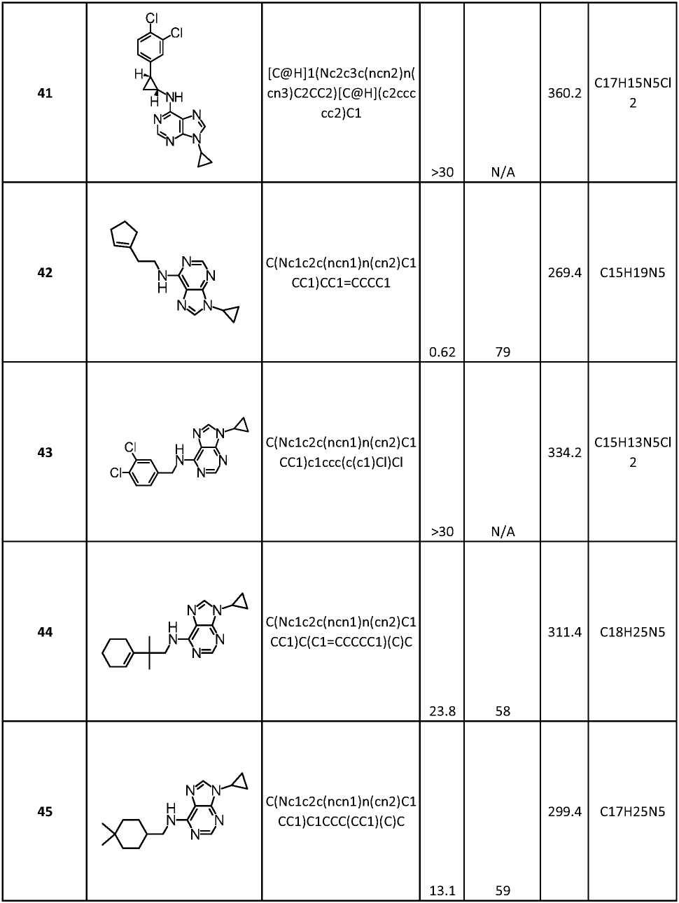

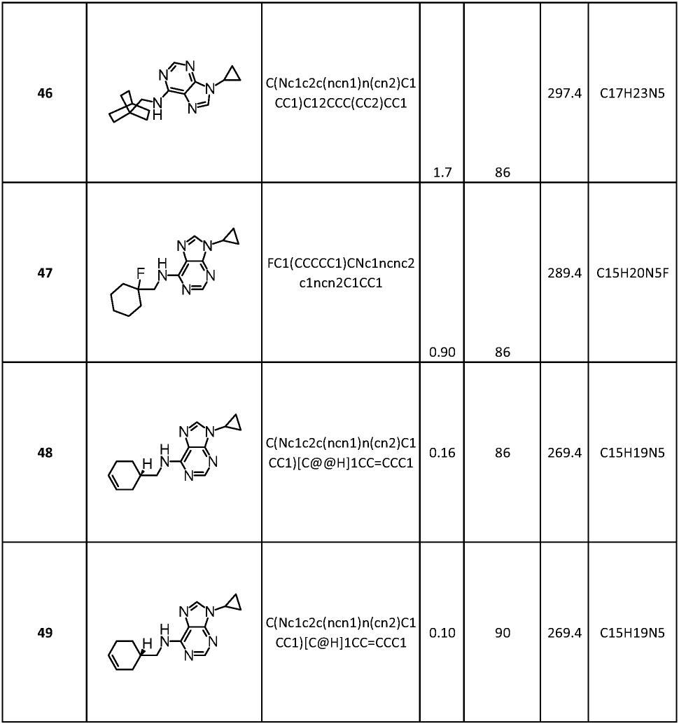

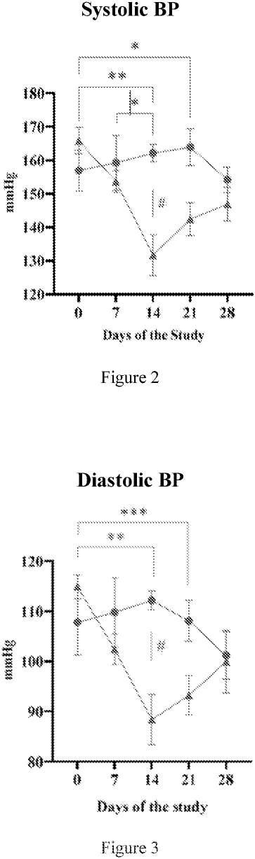

Systolic and diastolic blood pressure values were recorded, Significant difference between systolic blood pressure values in Compound 49-treated group (A) and vehicle-treated group (•) was registered at Day 14 of the study (see FIG. 2). Also, considerable decrease of systolic blood pressure was recorded during the Study in the Compound 49-treated group Diastolic blood pressure values were altered in the same manner: significant difference between the treated group and vehicle-treated group was pronounced on Day 14 (FIG. 3).

FIG. 2. Systolic BP values (mean per group ± SE) in aged male Wistar rats during the study. Statistical differences calculated using Tukey’s multiple comparisons test at the level of p < 0.05, p < 0.01 were given with asterisks * and ** within the Compound 49-treated group, and difference between the groups calculated using Sidak’s multiple comparisons test at the level of p < 0.05 were given with hash #, respectively. Circles (•) represent the vehicle-treated, control group and the triangles (▲) represent the drug-treated group.

FIG. 3: Diastolic BP values (mean per group ± SE) in aged male Wistar rats during the study. Statistical differences calculated using Tukey’s multiple comparisons test at the level of p < 0.05, p < 0.01 were given with asterisks * and ** within the Compound 49-treated group, and difference between the groups calculated using Sidak’s multiple comparisons test at the level of p < 0.05 were given with hash #, respectively. Green circles represent the vehicle-treated group and the red triangles are the drug-treated group.

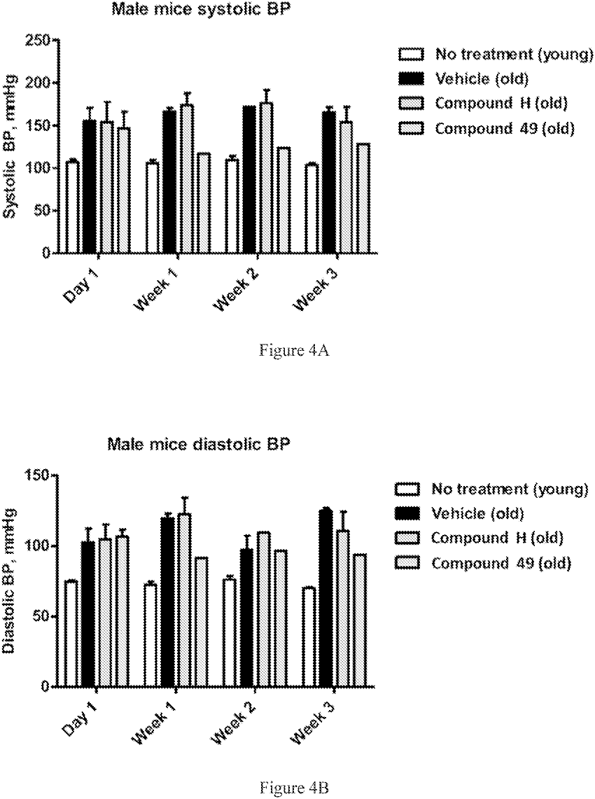

FIGS. 4A-4D illustrate, respectively systolic blood pressure (4A), diastolic blood pressure (4B), mean blood pressure (4C), and grip strength (4D) in male mice treated with illustrative Compound 49.

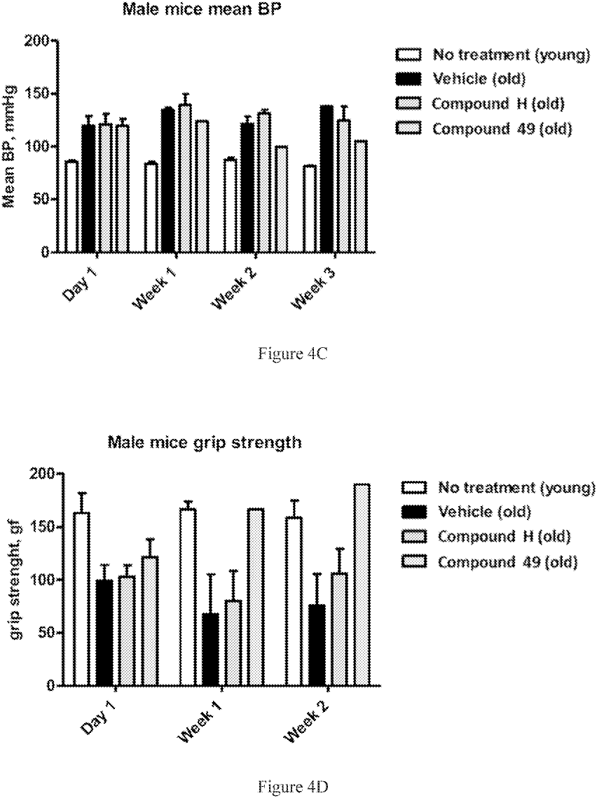

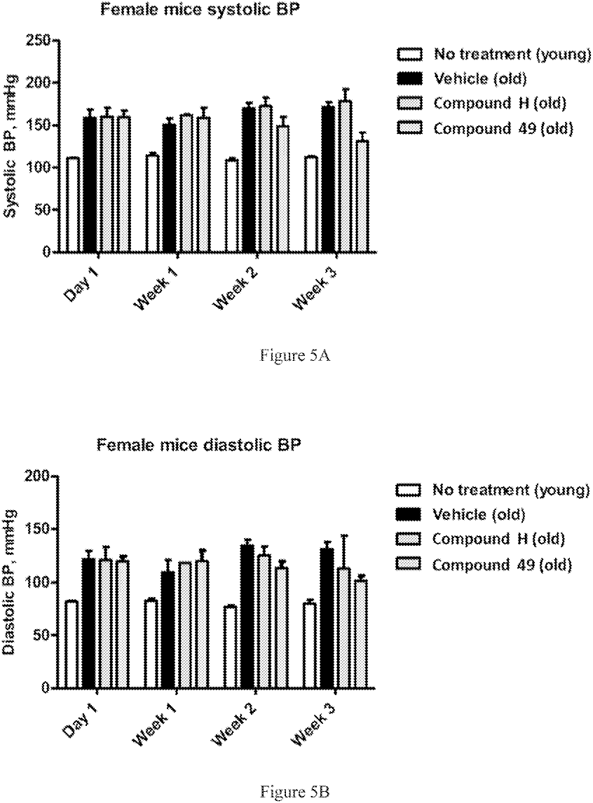

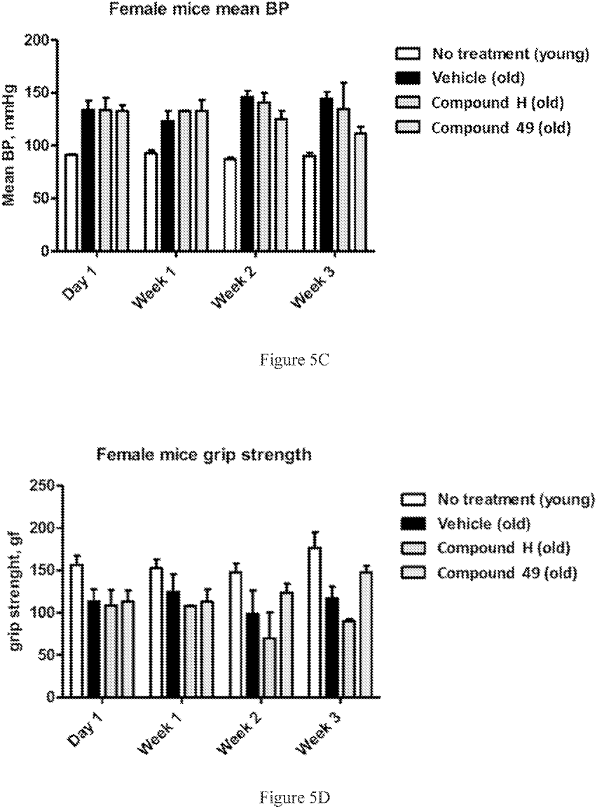

FIGS. 5A-4D illustrate, respectively systolic blood pressure (5A), diastolic blood pressure (5B), mean blood pressure (5C), and grip strength (5D) in female mice treated with illustrative Compound 49.

Compound 49 reduced systolic, diastolic, and mean blood pressure and increased grip strength (compared to untreated and treated (with existing compound H), aged control male and female mice) in one or more of the relevant time frames.

### Conclusion

While the foregoing detailed description makes reference to specific exemplary embodiments, the present disclosure may be embodied in other specific forms without departing from its spirit or essential characteristics. Accordingly, the described embodiments are to be considered in all respects only as illustrative and not restrictive. For instance, various substitutions, alterations, and/or modifications of the inventive features described and/or illustrated herein, and additional applications of the principles described and/or illustrated herein, which would occur to one skilled in the relevant art and having possession of this disclosure, can be made to the described and/or illustrated embodiments without departing from the spirit and scope of the disclosure as defined by the appended claims. Such substitutions, alterations, and/or modifications are to be considered within the scope of this disclosure.

The scope of the invention is, therefore, indicated by the appended claims rather than by the foregoing description. The limitations recited in the claims are to be interpreted broadly based on the language employed in the claims and not limited to specific examples described in the foregoing detailed description, which examples are to be construed as non-exclusive and non-exhaustive. All changes which come within the meaning and range of equivalency of the claims are to be embraced within their scope.

It will also be appreciated that various features of certain embodiments can be compatible with, combined with, included in, and/or incorporated into other embodiments of the present disclosure. For instance, systems, methods, and/or products according to certain embodiments of the present disclosure may include, incorporate, or otherwise comprise features described in other embodiments disclosed and/or described herein. Thus, disclosure of certain features relative to a specific embodiment of the present disclosure should not be construed as limiting application or inclusion of said features to the specific embodiment.

In addition, unless a feature is described as being requiring in a particular embodiment, features described in the various embodiments can be optional and may not be included in other embodiments of the present disclosure. Moreover, unless a feature is described as requiring another feature in combination therewith, any feature herein may be combined with any other feature of a same or different embodiment disclosed herein. It will be appreciated that while features may be optional in certain embodiments, when features are included in such embodiments, they can be required to have a specific configuration as described in the present disclosure.

Likewise, any steps recited in any method or process described herein and/or recited in the claims can be executed in any suitable order and are not necessarily limited to the order described and/or recited, unless otherwise stated (explicitly or implicitly). Such steps can, however, also be required to be performed in a specific order or any suitable order in certain embodiments of the present disclosure.

Furthermore, various well-known aspects of illustrative systems, methods, products, and the like are not described herein in particular detail in order to avoid obscuring aspects of the example embodiments. Such aspects are, however, also contemplated herein.

We claim:

1. A compound according to Formula I: 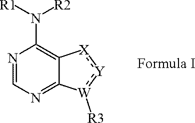
or a stereoisomer, tautomer or pharmaceutically acceptable salt thereof, wherein:
X is N, S, or C—R₄;
Y is N or C—R₄;
each R₄ is, independently, H or C1-C5 alkyl;
W is N, S, or C;
the bond between X-Y is a single bond or a double bond;
the bond between Y-W is a single bond or a double bond;
R₁ is H, CH₃, or, together with R₂, forms substituted or unsubstituted heterocyclic amine;
R₂ is:
(a) (CH₂)_{Z}—(CR₅R₆)_{V}—R₇, wherein:
Z is an integer from 0-2,
V is an integer from 0-2,
R₅ is H or CH₃;
R₆ is H or CH₃; and
R₇ is selected from the group consisting of:
(i) saturated or unsaturated C3-C8 substituted or unsubstituted cycloalkyl or bicycloalkyl (e.g., bicyclo octane, preferably, bicyclo(2.2.2)octane), optionally substituted at one or more (ring) positions and each (ring) substituent is selected from the group consisting of halo, aryl (phenyl or benzyl), or branched or unbranched C1-C3 alkyl;
(ii) substituted or unsubstituted aryl (phenyl or benzyl), optionally substituted at one or more (1 or 2) (ring) positions and each (ring) substituent is selected from the group consisting of halo or branched or unbranched C1-C3 alkyl; or (iii) branched or unbranched C1-C3 alkyl (e.g., isopropyl);
(b) C3-C7 substituted or unsubstituted cycloalkyl (e.g., 2-phenylcyclopropyl), optionally substituted at one or more (ring) positions and each (ring) substituent is selected from the group consisting of substituted or unsubstituted aryl (phenyl or benzyl), branched or unbranched C1-C3 substituted or unsubstituted alkyl; or
(c) together with R₁, forms substituted or unsubstituted heterocyclic amine; and
R₃ is selected from the group consisting of nothing, H, alkyl, cycloalkyl, aryl (phenyl or benzyl), nitrile, (CH₂)_{Z}CN, branched or unbranched C1-C3 substituted or unsubstituted alkyl, C3-C7 substituted or unsubstituted cycloalkyl, or aryl (phenyl or benzyl) substituted at one or more (ring) positions and each (ring) substituent is selected from the group consisting of branched or unbranched C1-C3 substituted or unsubstituted alkyl, halo, or nitrile.

2. The compound of claim 1, wherein R₁ is H and R₂ is (CH₂)_{Z}—(CR₅R₆)_{V}—R₇, wherein Z is an integer from 1-2, V is 0, and R₇ is (selected from (the group consisting of):
saturated or unsaturated C4-C7 cycloalkyl, optionally substitute at one or more (ring) positions with one or more methyl, preferably saturated C4-C6 cycloalkyl, optionally substitute at one or more (ring) positions with one or more methyl, more preferably unsubstituted saturated C4-C6 cycloalkyl or saturated C6 cycloalkyl optionally substituted at one or more (ring) position with one or more methyl or fluoro, still more preferably monounsaturated C5-C7 cycloalkyl, preferably 2-(1-cycloalkenyl or 4-(1-cycloalkenyl, more preferably 2-(1-cyclopentenyl), 2-(1-cyclohexenyl), 2-(1-cycloheptenyl), 4-(1-cyclopentenyl), 4-(1-cyclohexenyl), or 4-(1-cycloheptenyl);
bicycloalkyl, preferably bicyclo octane, more preferably, bicyclo(2.2.2)octane;
aryl (or phenyl or benzyl), preferably substituted at one or more (ring) positions with halo, preferably chloro, more preferably 3-chlorobenzyl or 2,3-dichlorobenzyl;
isopropyl; and/or
C3 cycloalkyl, preferably substituted with aryl (or phenyl or benzyl), preferably 2-phenylcyclopropyl.

3. The compound of claim 1, wherein R₁ is H and R₂ is (CH₂)_{Z}—(CR₅R₆)_{V}—R₇, wherein Z is 1, V is 1, R₅ is CH₃, R₆ is CH₃, and R₇ is monounsaturated cyclohexyl, preferably 2-(1-cyclohexenyl).R1 is H and R2 is (CH₂)_{Z}—(CR₅R₆)_{V}—R₇, wherein Z is 1, V is 1, R₅ is CH₃, R₆ is CH₃, and R₇ is monounsaturated cyclohexyl, preferably 2-(1-cyclohexenyl).

4. The compound of claim 1, wherein R₁ is CH₃ and R₂ is (CH₂)_{Z}—(CR₅R₆)_{V}—R₇, Z is 2, V is 0, and R₇ is cyclohexyl.

5. The compound of claim 1, wherein R₁ together with R₂, forms substituted or unsubstituted heterocyclic amine, preferably, substituted azepane, more preferably 4,4-diethylazepane. In some embodiments, R₁ and R₂ form a substituted or unsubstituted, saturated or unsaturated heterocyclic amine, preferably a substituted azepane, aziridine, azetidine, pyrrolidine, piperidine, or azocane, more preferably azepane, still more preferably 4,4-diethyl azepane.

6. The compound of claim 1, wherein:
X is N, Y is C—R₄, R₄ is H, W is N, the bond between X-Y is a double bond, and the bond between Y-W is a single bond, and wherein R₃ is preferably alkyl or cycloalkyl, more preferably ethyl or cyclopropyl;
X is C—R₄, Y is C—R₄, R₄ is H, W is N, the bond between X-Y is a double bond, and the bond between Y-W is a single bond, and preferably, wherein R₃ is H;
X is C—R₄, R₄ is H, Y is N, W is N, the bond between X-Y is a double bond, and the bond between Y-W is a single bond, and preferably, wherein R₃ is preferably (i) alkyl, more preferably methyl, (ii) substituted or unsubstituted aryl, phenyl or benzyl, still more preferably fluorophenyl or fluorobenzyl, or (iii) nitrile, preferably ethanenitrile or ethyl cyanide;
X is N or C—R₄, Y is C—R₄, each R₄ is, independently, H or CH₃, W is S, the bond between X-Y is a double bond, and the bond between Y-W is a single bond, and wherein R₃ is nothing;
X is C—R₄, Y is C—R₄, each R₄ is, independently, H or CH₃, W is S, the bond between X-Y is a double bond, and the bond between Y-W is a single bond, and wherein R₃ is nothing;
X is N, Y is C—R₄, R₄ is H, W is S, the bond between X-Y is a doble bond, and the bond between Y-W is a single bond, and wherein R₃ is nothing;
X is N, Y is C—R₄, R₄ is H, W is N, the bond between X-Y is a double bond, and the bond between Y-W is a single bond, and wherein R₃ is preferably C2-C3 alkyl or cycloalkyl, preferably, ethyl or cyclopropyl; or
X is S, Y is C—R₄, R₄ is H, W is C, the bond between X-Y is a single bond, and the bond between Y-W is a double bond, and wherein R₃ is preferably H.

7. The compound of claim 1, wherein X is N, Y is C—R₄, R₄ is H, W is N, the bond between X-Y is a double bond, the bond between Y-W is a single bond, R₁ is H, R₂ is (CH₂)_{Z}—(CR₅R₆)_{V}—R₇, wherein Z is 1, V is 0, and R₇ is (selected from (the group consisting of)) unsubstituted monounsaturated C6 cycloalkyl or unsubstituted cyclohexenyl, preferably 4-(1-cyclohexenyl), more preferably R-4-(1-cyclohexenyl) or S-4-(1-cyclohexenyl), and R₃ is cyclopropyl.

8. The compound of claim 1, wherein X is N, Y is C—R₄, R₄ is H, W is N, the bond between X-Y is a double bond, and the bond between Y-W is a single bond, R₃ is (selected from (the group consisting of)) alkyl or cycloalkyl, preferably ethyl or cyclopropyl, R₁ is H and R₂ is (CH₂)_{Z}—(CR₅R₆)_{V}—R₇, wherein Z is an integer from 1-2, V is 0, and R₇ is (selected from (the group consisting of)):
unsubstituted saturated C4-C6 cycloalkyl;
saturated C6 cycloalkyl substituted at one (ring) positions with fluoro, methyl, or dimethyl, preferably 1-fluorocyclohexyl, 1-methylcyclohexyl, or 4,4-dimethylcyclohexyl;
unsubstituted monounsaturated C5-C7 cycloalkyl, preferably 2-(1-cycloalkenyl or 4-(1-cycloalkenyl, more preferably 2-(1-cyclopentenyl), 2-(1-cyclohexenyl), 2-(1-cycloheptenyl), 4-(1-cyclopentenyl), 4-(1-cyclohexenyl), or 4-(1-cycloheptenyl);
bicyclo octane, more preferably, bicyclo(2.2.2)octane;
aryl (or phenyl or benzyl) substituted at one or two (ring) positions with halo, preferably chloro, more preferably 3-chlorobenzyl or 2,3-dichlorobenzyl;
isopropyl; and/or
C3 cycloalkyl substituted with aryl (or phenyl or benzyl), preferably 2-phenylcyclopropyl.

9. The compound of claim 1, wherein the compound is according to Formula Ia: 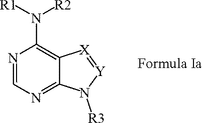
or a stereoisomer, tautomer or pharmaceutically acceptable salt thereof.

10. The compound of claim 9, wherein X is N, Y is CH, R₁ is H, R₂ is CH₂—R₈, R₈ is cyclohexenyl, preferably 2-(3-cyclohexenyl), and R₃ is cyclopropyl.

11. The compound of claim 10, wherein the compound is according to Formula 48 or Formula 49:

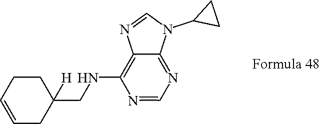 [SMILES: C1=CCC(CNc2ncnc3c2ncn3C2CC2)CC1]

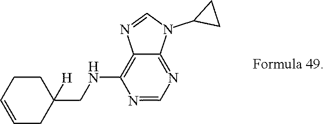 [SMILES: C1=CCC(CNc2ncnc3c2ncn3C2CC2)CC1]

12. A compound according to Formula II: 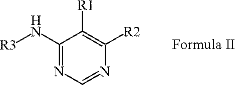
or a stereoisomer, tautomer or pharmaceutically acceptable salt thereof, wherein:
R₁ is H or NH₂;
R₂ is saturated or unsaturated, heterocyclic amine or heterocyclic diamine;
R₃ = (CH₂)₂—R₇, wherein R₇ is unsaturated cycloalkyl.

13. The compound of claim 12, wherein R₂ is saturated heterocyclic amine, preferably N-pyrrolidine or N-piperidine, or unsaturated heterocyclic diamine, preferably N-pyrazole.

14. The compound of claim 12, wherein, when R₁ is H, then R₂ is N-pyrrolidine or N-piperidine.

15. The compound of claim 12, wherein, when R₁ is NH₂, then R₂ is N-pyrazole.

16. The compound of claim 12, wherein R₇ is monounsaturated C6-C7 cycloalkyl, preferably cyclohexenyl, more preferably 2-(1-cyclohexenyl), or cycloheptenyl, more preferably 2-(1-cycloheptenyl).

17. The compound of claim 12, wherein the compound is according to one for Formulas IIa—IId— 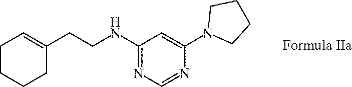 [SMILES: C1=C(CCNc2cc(N3CCCC3)ncn2)CCCC1] 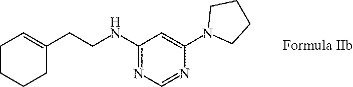 [SMILES: C1=C(CCNc2cc(N3CCCC3)ncn2)CCCC1] 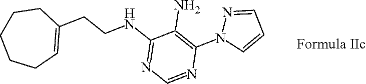 [SMILES: Nc1c(NCCC2=CCCCCC2)ncnc1-n1cccn1] 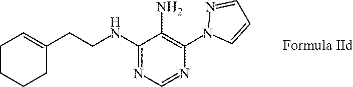 [SMILES: Nc1c(NCCC2=CCCCC2)ncnc1-n1cccn1]
.

18. A compound according to Formula III: 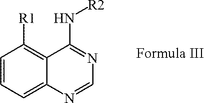
or a stereoisomer, tautomer or pharmaceutically acceptable salt thereof, wherein:
R₁ is halo; and
R₂ is (CH₂)₂—R₇, wherein R₇ is unsaturated cycloalkyl.

19. The compound of claim 18, wherein R₁ is fluoro and R₇ is unsaturated cycloalkyl, preferably monounsaturated cycloalkyl, more preferably cyclohexenyl, still more preferably 2-(1-cyclohexenyl), most preferably, wherein the compound is according to Formula IIIa:

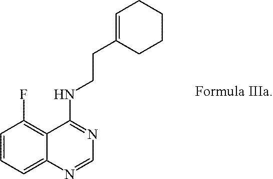 [SMILES: Fc1cccc2ncnc(NCCC3=CCCCC3)c12]

20. A composition, comprising:
the compound of claim 1; and
a pharmaceutically-acceptable carrier.

20-25. (canceled)
